(19) United States
(12) Patent Application Publication
(10) Pub. No.: US 2023/0337665 A1
(43) Pub. Date: Oct. 26, 2023

(54) PERACID BOOSTER COMPOSITIONS AND METHODS OF USING SAME

(71) Applicant: Sterilex, LLC, Hunt Valley, MD (US)

(72) Inventors: Walter Daniel Cummings, New Freedom, PA (US); Gregory Rosenberg, Rochester, NY (US)

(21) Appl. No.: 18/214,650

(22) Filed: Jun. 27, 2023

Related U.S. Application Data
(63) Continuation-in-part of application No. 18/073,242, filed on Dec. 1, 2022.
(60) Provisional application No. 63/463,474, filed on May 2, 2023.
(60) Provisional application No. 63/425,537, filed on Nov. 15, 2022.
(60) Provisional application No. 63/293,962, filed on Dec. 27, 2021.

(51) Int. Cl. A01N 37/16; A01N 37/06; A01N 37/02; A01N 25/16; A01P 1/00; A61L 2/18

(52) U.S. Cl. CPC A01N 37/16; A01N 37/06; A01N 37/02; A01N 25/16; A01P 1/00; A61L 2/18

(57) ABSTRACT

Disclosed are compositions and methods for disinfecting, sterilizing, and/or sanitizing a surface associated with human or veterinary medical use, pharmaceutical manufacturing, or cosmetic manufacturing. The methods include contacting a surface with a peracid-containing composition and a peracetic acid (PAA) boosting composition. The methods may be useful for deactivating, removing, and/preventing a virus, an endospore, a bacterium, and combinations thereof.

### CROSS-REFERENCE TO RELATED APPLICATIONS

This application is a continuation-in-part of U.S. application Ser. No. 18/073,242, filed Dec. 1, 2022, which claims priority to and benefit of U.S. Provisional Application Ser. No. 63/293,962, filed Dec. 27, 2021 and U.S. Provisional Application Ser. No. 63/425,537, filed Nov. 15, 2022, and further claims priority to and benefit of U.S. Provisional Application Ser. No. 63/463,474 filed on May 2, 2023, the contents of each are incorporated in their entirety for all purposes.

### BACKGROUND

Peracid containing disinfectants and/or sanitizers (collectively, “peracid disinfectants/sanitizers” or “PA disinfectants/sanitizers”) are used in a variety of settings to control bacteria and other pathogens. Such PA disinfectants/sanitizers can be sold in the form of an aqueous liquid concentrate that is intended for dilution with water to generate a use solution that is applied to a hard surface to be disinfected or sanitized. Some concentrated PA disinfectants/sanitizers may be sold as a kit with a separate solution to be used to dilute the concentrated PA disinfectants/sanitizers to provide for a use solution to be applied to a hard surface.

The dilution of concentrated PA disinfectants/sanitizers to produce use solutions may depend upon the intended use thereof. For example, a concentrated peracetic acid (“PAA”) solution that is to be used to sanitize a hard surface, may be diluted to generate a use solution that contains PAA from about 140 to about 200 parts per million (hereinafter, “ppm”). In a further example, a concentrated PAA solution that is to be used to disinfect a hard surface, may be diluted to generate a use solution that contains PAA from 250 to about 450 ppm. While use solutions of known PAA disinfectant/sanitizers may effectively kill/control bacteria at these PAA concentrations, to additionally, or alternatively, act as a sterilant that also kill viruses and/or spores, it is believed that a significantly higher concentration of PAA in use solutions would be needed. Moreover, it is believed that known use solutions of PAA disinfectant/sanitizers are not generally found to be effective in penetrating, sterilizing and/or removing biofilms from hard surfaces without being in contact with the biofilms for 20 minutes or more and/or by comprising 800 ppm or more of total peracid (hereinafter, “PA”).

Literature in the art suggests that to be sporicidal, use solutions of PAA disinfectant/sanitizers should contain at least 2,000 ppm of PAA, which is believed to be as much as 14 times the amount of PAA that would be required for a use solution that could be used to sanitize a hard surface. However, preparing and applying solutions containing such relatively high levels of PAA may result in drawbacks including, but not limited to, those that are described in U.S. Pat. Pub. No. 2016/0345576 to Rovison et al. Because PAA, and other peracids, are known to be a highly corrosive material, increasing levels of PAA and/or other peracids in concentrated PA disinfectants/sanitizers that are to be diluted to provide for use solutions that are effective as a sterilant and/or for sterilizing and/or removing biofilms from hard surfaces, may result in undesirable side effects. For example, the cost and complexity associated with making, packaging, shipping and handling of such PAA concentrates can be significantly increased. Moreover, such PAA solutions may damage the hard surfaces to which they are applied.

U.S. Pat. Pub. No. 2018/0187129 to Traistaru et al. (hereinafter, “U.S. 129”) discloses acid detergent compositions that are said to be suited for removing soils, especially milk soils, from clean-in-place systems, such as those commonly used in the dairy and food processing industries. U.S. '129 indicates that its detergent compositions generally comprise an acidic component containing an inorganic acid or alkanesulfonic acid alone or optionally in combination with an organic acid or another acid, e.g., glycolic acid, which is different from the first inorganic or alkanesulfonic acid. U.S. '129 further indicates that its detergent compositions may be concentrated and that those detergent compositions can be diluted with water to make use solutions. U.S. '129 further indicates that the disclosed acid detergent compositions may include organic and inorganic peroxygen bleaches and peracids, such as hydrogen peroxide, and activated hydrogen peroxides like peracetic acid. U.S. '129 indicates that in certain embodiments, detergent concentrates exhibit a pH of less than 2, of less than 1, or from about −1 to about 1, or from about −0.7 to about 0.4, and in certain embodiments, the pH of the use solution is from about 0.1 to about 5, from about 1 to about 4, or from about 2.1 to about 2.5.

U.S. Pat. Pub. No. 2019/0045789 to Daigle (hereinafter, “U.S. '789”) discloses an antimicrobial composition comprising lauric arginate ethyl ester (LAE) and hydrogen peroxide, and the use of this composition for disinfecting and sanitizing different types of surfaces such as food products, human skin and hard surfaces, as well as a method of stabilizing the composition by including a sequestering agent (citrate salt and/or phosphate salt). U.S. '789 indicates that its composition may be a concentrate composition that is diluted with tap water on site. U.S. '789 indicates that the antimicrobial composition may optionally include one or more additional antimicrobial ingredients for specific application on biopolymer or surface, for example, organic acid. U.S. '789 opines that an inhibitory mechanism of organic acids occurs via diffusion of undissociated acids across the membrane barrier of a cell and subsequent dissociation in the cytoplasm of a cell, which causes a number of physiological disruptions such as reduced intracellular pH.

U.S. Pat. Pub. No. 2021/0259245 to Li et al. (hereinafter, U.S. '245) discloses highly acidic, stabilized peroxycarboxylic acid compositions having both improved antimicrobial efficacy in comparison to conventional peroxyoctanoic acid and peroxyacetic acid compositions for sanitizing applications, and improved transport and shipping stability. U.S. '245 indicates that its low odor and low/no VOC compositions having dual functionality as both acid wash and sanitizing compositions are disclosed. U.S. '245 also indicates that an embodiment of the invention is a composition comprising: a C1-C22 carboxylic acid; a C1-C22 percarboxylic acid; hydrogen peroxide; and a stabilizing agent, wherein the stabilizing agent is a picolinic acid or a compound having the following Formula (IA):

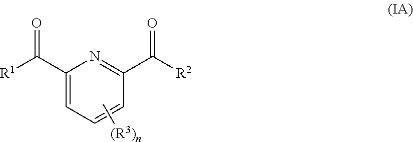

U.S. '245 indicates that its invention further provides peroxycarboxylic acid stabilizing compounds suitable for use in compositions having extreme ratios of peracid to hydrogen peroxide, wherein the concentration of the peroxyacids greatly exceed the hydrogen peroxide.

U.S. Pat. Pub. No. 2021/0087499 to Marchand et al. (hereinafter, U.S. '499) discloses peracetic compositions, methods and kits for removing biofilms from an enclosed surface. U.S. '499 further discloses compositions comprising (i) peracetic acid; (ii) at least one secondary acid; and (iii) a non-foaming surfactant. A method disclosed in U.S. '499 comprises contacting, preferably for at least 5 min, a surface with such a peracetic such composition. Another method disclosed in U.S. '499 comprises circulating such a composition into a piping system for a period of time providing for disruption and/or removal of a biofilm. U.S. '499 discloses kits comprising bottles of concentrated solutions to be mixed and diluted with water before use. U.S. '499 indicates that compositions, methods and kits are particularly useful to remove bacterial biofilms from enclosed surfaces such as piping systems that are commonly used in the food and beverages industries. U.S. '499 discloses that according to one aspect, the invention concerns a kit for disruption of biofilm on, and/or removal of biofilm from, a surface, the kit comprising: a first bottle comprising a first concentrated solution, the first concentrated solution comprising about 3% w/w to about 30% w/w peracetic acid, and about 0.001% w/w to about 5% w/w of a surfactant; and a second bottle comprising a second concentrated solution, the second concentrated solution comprising about 1% w/w to about 50% w/w of at least one secondary acid. U.S. '499 indicates that in one embodiment, a method comprises circulating into the piping system a composition containing 800 ppm of PAA for a period of time providing for successful disruption and/or removal from the biofilm. Particularly, the circulating may be carried out for at least 5 minutes, or least 10 minutes, or at least 15 minutes, or at least 20 minutes, or at least 30 minutes or more. U.S. '499 discloses that in another embodiment, a ready to use solution comprising at least 400 ppm of PAA may be placed in contact with a biofilm on a hard surface for 20 minutes. U.S. '499 indicates that while it is within the skill of those in the art to determine an acceptable period of time providing for a desired efficacy, typically a longer period of time may be preferred to achieve better biofilm removal.

U.S. Pat. Pub. No. 2017/0128605 to Franciskovich et al. (hereinafter, U.S. '605) discloses a two-part liquid composition for cleaning and disinfecting a substrate, which may be a medical device, for example, an endoscope. U.S. '605 indicates that the two-part liquid composition comprises: (A) a disinfectant medium comprising peracetic acid; and (B) a supplemental medium comprising a non-enzymatic cleaner, a corrosion inhibitor, and a chelator. The supplemental medium (B) may further comprise an enzymatic cleaner, a surfactant, a buffer, a pH modifier, or a mixture of two or more thereof. U.S. '605 indicates that the non-enzymatic cleaner comprises an alkanol amine, an alcohol ethoxylate, an alkyl glucoside, an alkylene glycol, an alkyl diproprionate, an alkyl dialkylamine oxide, or a mixture of two or more thereof. U.S. '605 discloses that the pH of the disinfectant medium (A) may be in the range of from about 1 to about 8, or from about 3 to about 6, and that the pH of the supplemental medium (B) may be in the range of from about 6 to about 14, or from about 6 to about 9.

U.S. Pat. Pub. No. 2016/0345576 to Rovison et al. (hereinafter, U.S. '576) discloses anti-microbial compositions that are purported to be useful against a wide range of microorganisms that are undesirable on a wide variety of materials, including food, food contact and non-food contact surfaces, and surfaces in industrial, recreational, health care, and other institutional environments. According to U.S. '576, the anti-microbial compositions more particularly comprise peracetic acid in combination with a) citric acid or a salt and b) salicylic acid or a salt in aqueous solution. Additionally, U.S. '576 indicates that the disclosed invention further includes a kit comprising: a. an aqueous peracetic acid solution; and b. a mixture comprising salicylic acid and citric acid or their respective salts in proportionate ratios ranging from about 1:5 to 1:1 by weight respectively and in a solution matrix acting as solvent of glacial acetic acid (70-90%) and water (30-5%). U.S. '576 further discloses that peracetic acid is known to be a useful antimicrobial agent. U.S. '576 further discloses that PAA's antimicrobial effect is best obtained with high concentrations (generally greater than 100 parts per million (ppm)). U.S. '576 opines that at these concentrations, the PAA has an overbearing odor, sometimes causes oxidative damage to foodstuffs and surfaces to which it is applied, and present hazards to persons handling the materials. U.S. '576 indicates that U.S. Pat. No. 4,051,058 discloses aqueous solutions of peracetic acid in concentrations of 0.5% to 20% by weight for use in sanitizing and disinfecting applications. U.S. '576 further indicates that U.S. Pat. No. 6,617,290 discloses the use of acidifying agents classified as GRAS (Generally Regarded As Safe) for use as food additives in preparations for cleaning and sanitizing food contact and non-food contact surfaces, noting that these agents include citric and lactic acids. U.S. '576 further indicates that U.S. Pat. No. 6,475,967 discloses a light duty antibacterial liquid detergent displaying foaming and grease-cutting as well as low corrosive properties, wherein the aqueous composition comprises hydroxyl-containing organic acid(s), a peracetic acid, and other components including various surfactants, and polyethylene glycol.

A need remains for PAA disinfectants/sanitizers that can be used to kill bacteria, viruses and/or spores on a variety of hard surfaces and in a variety of settings, in particular for reducing, deactivating, removing, and/or preventing a biofilm. A biofilm is a biological conglomerate containing pathogens, such as bacteria and other microorganisms, embedded in a matrix of exopolymers and macromolecules, that can further contain other microorganisms such as fungi, molds, algae, protozoa, archaea and mixtures of these microorganisms. Biofilms are believed to form as a result of microorganisms establishing on a surface, which then produce a protective extracellular polymeric matrix. Biofilms tend to form on surfaces in contact with water, providing a hydrated matrix of polysaccharides to provide structural protection from biocides, making biofilm more difficult to kill than other pathogens. The biomass accumulation is a buildup of microorganisms and/or extracellular substances including dirt and debris that become trapped in the biomass. Such biofilm biomasses are difficult to eradicate.

Biofilm masses may further harbor dangerous bacterial species. Control of pathogens such as Clostridium difficile (C.diff), for example, is a well-recognized need in the art, and such pathogens may be harbored in biofilms. For example, hospital acquired or associated infections are a significant cause of mortality and morbidity in the United States. (See, e.g., U.S. Pat. No. 10,255,466.) Such infections may be attributable to surface contamination, particularly for Methicillin resistant Staphylococcus aureus (MRSA) and C. cliff. Hospital acquired infections, for example, are believed to frequently result from cross contamination via health care personnel either by direct patient contact or by touching contaminated environmental surfaces. While well-recognized in the hospital setting, there is further a need to minimize, reduce, or largely eliminate the possibility of pathogens such as C.diff and other Clostridium species from surfaces that may serve as a vehicle for infection, such as medical/hospital surfaces, including hospital equipment and devices, as well as pharmaceutical manufacturing surfaces and equipment, as well as and cosmetic manufacturing surfaces and equipment, all of which would benefit from methods that can be used to minimize the presence of harmful bacterial.

Thus, it would be beneficial for such PAA disinfectants/sanitizers to also be effective in reducing, deactivating, removing, and/or preventing bacterial biofilms. It would also be beneficial for such PAA disinfectants/sanitizers to be useful in environmental cleaning, cleaning in place (hereinafter “CIP”) and/or in cleaning out of place (hereinafter, “COP”) applications in a variety of settings. It would also be beneficial for the efficacy of PAA in existing PAA disinfectants/sanitizers to be increased without having to add more PAA thereto including, but not limited to, increasing efficacy of PAA disinfectants/sanitizers such that they may penetrate biofilms and kill and/or remove biofilms from a hard surface. It would further be beneficial for a composition that can be added to a wide variety of existing PAA disinfectant/sanitizers to increase the efficacy of the PAA in existing PAA disinfectants/sanitizers, rather than formulating a PAA disinfectants/sanitizers to increase efficacy while reducing shelf stability (e.g., by adding surfactants to them). It would further be beneficial to provide a composition that can be added to PAA disinfectants/sanitizers and the resulting mixture diluted to provide for the intended end use, e.g., disinfection, sterilization, sanitization and/or biofilm penetration, kill, and/or removal.

A need remains for concentrates and use solutions of PA disinfectants/sanitizers that can be used to kill bacteria, viruses and/or spores on a variety of hard surfaces and in a variety of settings. It would be beneficial for such PA disinfectants/sanitizers to also be effective in penetrating, sterilizing and/or removing bacterial biofilms from hard surfaces. It would also be beneficial for the efficacy of PA in existing PA disinfectants/sanitizers to be increased without having to add more PA thereto including, but not limited to, increasing efficacy of PA disinfectants/sanitizers such that they may penetrate biofilms and act as sterilants and/or kill and/or remove biofilms from a hard surface. It would further be beneficial for there to be a single composition that can be added to a wide variety of existing PA disinfectant/sanitizer concentrates and/or use solutions of PA disinfectant/sanitizers to increase the efficacy of the PA contained therein, rather than formulating new concentrates and/or use solutions to increase efficacy while potentially reducing shelf stability (e.g., by adding surfactants to them). It would further be beneficial to provide for compositions that can be added to existing concentrates of PA disinfectants/sanitizers, so that the resulting use solutions provide for a wide variety of intended uses including, but not limited to, disinfection, sterilization, sanitization, biofilm penetration, biofilm sterilization, removal of biofilm and combinations thereof, from hard surfaces. It would also be beneficial for such PA disinfectants/sanitizers to be useful in environmental cleaning, cleaning in place (“CIP”) and/or in cleaning out of place (hereinafter, “COP”) applications in a variety of settings.

While a variety of PA disinfectants/sanitizers have been made and used, it is believed that no one prior to the inventors has made or used an invention as described herein.

The instant disclosure addresses one or more of the aforementioned needs in the art.

### BRIEF SUMMARY

Disclosed herein are peracetic acid (PA) booster compositions and methods of using same. Exemplary PA boosters can be foaming or low or non-foaming. Exemplary foaming PA boosters are aqueous and comprise a primary organic acid selected from: glycolic acid, benzoic acid, fumaric acid, caprylic acid, lactic acid, proprionic acid, a salt of any one of the foregoing organic acids and combinations thereof. Exemplary PA boosters further comprise an ancillary organic acid that is different from the primary organic acid, the ancillary organic acid having a carbon chain length of C6 or less, and at least one pKa of from about 2 to about 7. Exemplary PA boosters may further comprise a biodispersant comprising an anionic surfactant. Exemplary anionic surfactants may be selected from: alkyl sulfosuccinate, alkyl sulfoacetate, alkylamide hydrolysate; a metal salt of any one the foregoing, and combinations thereof. Exemplary PA boosters may further comprise a chelant and a pH buffering agent. Exemplary PA boosters may have a pH of from about 3 to about 4. Exemplary PA boosters may be substantially free of components selected from: peracids; peroxides selected from organic peroxides, inorganic peroxides and combinations thereof; inorganic acids; and combinations thereof.

The disclosed compositions may be provided as a kit. Exemplary kits comprise (a) a first container containing a foaming or low/foaming PA booster and (b) a second container containing an aqueous composition comprising one or more PAs.

Exemplary methods may comprise boosting efficacy of an aqueous use solution comprising, consisting of, or consisting essentially of, a concentrated disinfectant/sanitizer comprising one or more peracids diluted with water, so that the aqueous use solution can effectively achieve an anti-microbial effect, for example, penetrating a biofilm and killing bacteria contained therein. Exemplary methods may comprise making a disinfectant/sanitizer use solution by mixing together a concentrated peracid containing disinfectant/sanitizer with water and an exemplary foaming or no/low foaming PA booster, such that the efficacy of the resulting PA use solution is boosted by from about a 2 log₁₀ to about 5 log₁₀ cycles in comparison to a use solution comprising the same concentrated peracid containing disinfectant/sanitizer and water.

### BRIEF DESCRIPTION OF THE DRAWINGS

This application file contains at least one drawing executed in color. Copies of this patent or patent application publication with color drawing(s) will be provided by the Office upon request and payment of the necessary fee.

It is believed that the present invention will be better understood from the following description of certain examples taken in conjunction with the accompanying drawings:

FIG. 1 is a graphical representation of biofilm treatment.

FIG. 2 is a graphical representations of a recirculating liquid foam tester (RALF).

FIG. 3 depicts apparatuses used for testing of PAA+/− booster.

FIG. 4 depicts PAA vs. PAA with PA Booster CDC Reactor Results.

FIG. 5 depicts confocal images of Pseudomonas aeruginosa biofilm on after a 10-minute treatment at 20° C. with A) Hard water 1×, B) PAA with an exemplary PA Booster 1×, C) Hard water 3×, and D) PAA with PA Booster 3×.

FIG. 6. depicts PAA vs. PAA with PA Booster Sporicidal Results for Bacillus subtilis.

FIG. 7 depicts PAA vs. PAA with PA Booster Sporicidal Results for Bacillus subtilis, via clean in place (CIP) application.

FIG. 8 depicts C. sporogenes spore recovery after a ten-minute contact time at room temperature (RT), foam combined data.

FIG. 9 depicts C. sporogenes spore recovery from carriers after a 10-minute contact time at 50° C., via clean-in-place (CIP) application.

The drawings are not intended to be limiting in any way, and it is contemplated that various aspects of the invention may be carried out in a variety of other ways, including those not necessarily depicted in the drawings. The accompanying drawings that are incorporated in and form a part of the specification illustrate several aspects of the present invention, and together with the description serves to explain the principles of the invention.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive. It is believed that the present invention will be better understood from the following description of certain examples taken in conjunction with the accompanying drawings.

All percentages, parts, and ratios as used herein, are by weight of the total composition, unless otherwise specified. All such weights, as they pertain to listed ingredients, are based on the active level and, therefore, do not include solvents or by-products that may be included in commercially available materials, unless otherwise specified.

All references, including patent applications, patent publications and non-patent literature, which are referred to in the present specification are incorporated by reference herein, unless it is expressly indicated that they are not incorporated by reference herein.

Numerical ranges as used herein are intended to include every number and subset of numbers within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9 and so forth.

All references to singular characteristics or limitations of the present disclosure shall include the corresponding plural characteristic or limitation, and vice versa, unless otherwise specified or clearly implied to the contrary by the context in which the reference is made. E.g., as used herein and in the appended claims, the singular forms “a,” “and,” and “the” include the plural of the referenced term unless the context clearly dictates otherwise. Thus, for example, reference to “a method” includes a plurality of such methods and reference to “a dose” includes reference to one or more doses and equivalents thereof known to those skilled in the art, and so forth.

All combinations of method or process steps as used herein can be performed in any order, unless otherwise specified or clearly implied to the contrary by the context in which the referenced combination is made. The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive. The methods may comprise, consist of, or consist essentially of the elements of the compositions and/or methods as described herein, as well as any additional or optional element described herein or otherwise useful in the cleaning, disinfecting, sanitizing of surfaces, particularly for the removal or cleaning of a bacteria or a biofilm.

Unless otherwise noted, terms are to be understood according to conventional usage by those of ordinary skill in the relevant art. In case of conflict, the present document, including definitions, will control.

“About” or “approximately” as used herein, means within an acceptable error range for the particular value as determined by one of ordinary skill in the art, which will depend in part on how the value is measured or determined, e.g., the limitations of the measurement system. For example, “about” can mean within 1, or more than 1 standard deviation, per the practice in the art. Alternatively, “about” can mean a range of up to 15%, or up to 10%, or up to 5%, or up to 1%, or up to 0.5%, of a given value. Where particular values are described in the application and claims, unless otherwise stated the term “about” meaning within an acceptable error range for the particular value should be assumed.

“Biofilm” as used herein, means complex microbial communities characterized by cells attached to surfaces, interfaces, and/or each other, wherein the cells are embedded in a matrix of extracellular polymeric substances (“EPS”) of microbial origin.

“CIP application” or “Clean in Place application” as used herein, refers to an application of a use composition as described herein, for effectively treating hard surfaces (e.g., equipment and/or systems that handle (e.g., circulate and/or process)) a flowable substance (for example, liquids, emulsions, flowable solids (e.g., solid particulates) and combinations thereof). Such equipment and/or systems may not require complete disassembly to treat interior surfaces thereof, which may come into contact with the flowable substance. Exemplary interior surfaces of equipment and/or systems that may come into contact with flowable substances include, but are not limited to, vessels (e.g., tanks), filters, pumps, pipes, hoses, associated fittings and combinations thereof.

“Clear” or “clear to the naked eye” as used herein, means appearing clear to a human having 20/20 vision, without the use of any special imaging equipment.

“COP application” or “Clean Out of Place application” as used herein, refers to an application of use compositions as described herein, to effectively treat surfaces that can be soaked in the use compositions or to which the use solutions can be readily applied without substantial disassembly of equipment and/or systems. Such surfaces may include those having a single dimension, which may in turn include, but are not limited to, surfaces selected from plates, test coupons, countertops, walls, floors, thresholds and combinations thereof.

“Environmental application” as used herein, refers to an application of use compositions as described herein, to effectively treat exposed surfaces on equipment, floors, countertops, walls, floors, thresholds, drains, etc. For example, environmental application may include treatment of exposed surfaces that come into contact with food and/or beverages such as slicers, conveyors, food processing machines/equipment, etc.

“Disinfectant” as used herein, refers to a substance or a mixture of substances (including solutions) that destroy or irreversibly inactivate bacteria, fungi and viruses, but not necessarily bacterial spores, in on a hard surface.

As used herein, the term “effective amount” means the amount of one or more active components that is sufficient to show a desired effect.

“Effectively treat” as used herein, means to reduce microbial growth on a surface to which an exemplary use solution has been applied, as compared to the microbial growth on a comparable control surface to which use solution has not been applied. “Effective treatment” of a surface may include successful treatment of a hard surface, wherein the treatment is selected from: cleaning, sterilizing, disinfecting, sanitizing biofilm, killing biofilm, removing biofilm and combinations thereof.

“Foaming” as used herein, refers to a foam profile of at least about 9 inches of foam generation in under 30 seconds and dissipation of the foam in greater than or equal to 5 min, as determined via a foaming evaluation as described hereinbelow.

“Low foaming” as used herein, refers to a foam profile of at least about 5 inches of foam generation in under 10 min. and dissipation of the foam in 2 min. or less, as determined via the foaming evaluation as described hereinbelow.

“No foaming” as used herein, refers to a foam profile of less than or equal to 1 in. of foam generation in under 10 min. and dissipation of the foam in 30 seconds or less, as determined via the foaming evaluation as described hereinbelow.

“Low/No foaming” as used herein, encompasses both low foaming and no foaming compositions as described herein.

“PAA disinfectant/sanitizer” as used herein, refers to a composition that contains peracetic acid (“PAA”) and that acts as a disinfectant and/or sanitizer. “Disinfecting/sanitizing” refers to the act of disinfecting and/or sanitizing. “Disinfection/sanitization” refers to the characteristic of disinfecting and/or sanitizing.

“Sanitizer” as used herein, means a substance or a mixture of substances (including solutions) that reduce a bacterial population on a hard surface by significant numbers, (e.g., a 3 log₁₀ reduction) or more, but that does not destroy or eliminate all bacteria.

“Sterilant” as used herein, refers to a composition that destroys all viable forms of microbial life on a hard surface. As used herein, the term “sterilizing” means destroying substantially all, or all, microorganism, including, but not limited to bacteria, endospores, viruses, and combinations thereof.

“Substantially free” with respect to a component, class of components or combinations of components that has or have been specifically identified herein, means no effective amount of that specifically identified component, class of components or combinations of components, or from about 1 wt. % or less, from about 0.1 wt. % or less, or even from about 0.01 wt. % or less, or 0% (i.e., completely free) of the specifically identified component or class of components as specified herein. For example, “substantially free of PA,” or “PA free” as used herein, means from about 1 wt. % or less, from about 0.1 wt. % or less, or even from about 0.01 wt. % or less, or 0% (i.e., completely free) of one or more peracids. In a further example, “substantially free of PAA,” or “PAA free” as used herein, means from about 1 wt. % or less, from about 0.1 wt. % or less, or even from about 0.01 wt. % or less, or 0% (i.e., completely free) of PAA. In a further example, “substantially free of peroxides” as used herein, means from about 1 wt. % or less, from about 0.1 wt. % or less, or even from about 0.01 wt. % or less, or 0% (i.e., completely free) or peroxides selected from: organic peroxides (including salts of organic peroxides), inorganic peroxides (including salts of inorganic peroxides) and combinations thereof. In a further example, “substantially free of inorganic acids” as used herein, means from about 1 wt. % or less, from about 0.1 wt. % or less, or even from about 0.01 wt. % or less, or 0% (i.e., completely free) of inorganic acids.

“Peracid disinfectant/sanitizer use solution” or “PA disinfectant/sanitizer use solution” as used interchangeably herein, refers to a solution that is ready to be applied to a hard surface (i.e., is “ready to use”). A PA disinfectant/sanitizer use solution may be made prior to application to a hard surface, by diluting a concentrated peracid disinfectant/sanitizer with water and/or premade solutions. PA disinfectant/sanitizer use solutions and concentrated PA disinfectant/sanitizer solutions contain peracids selected from: peroxyacetic acid; peroxyoctanoic acid; peroxysulfonic acid; peroxyformic acid; and combinations thereof. PA disinfectant/sanitizer use solutions include, but are not limited to, peracetic acid use solutions (“PAA disinfectant/sanitizer use solutions”), which may be made prior to application to a hard surface, by diluting a concentrated peracetic acid disinfectant/sanitizer solution with water.

Log₁₀ reduction correlates to a 10-fold reduction, i.e., a 1-log₁₀=90% reduction. Table 1 shows stepwise log₁₀ reductions. The performance standard of log₁₀ reduction is commonly measured in terms of colony forming units (“CFUs”). Referencing Table 1, an 8.7-log₁₀ inoculum has 501187233.627 CFUs, and a 1-log₁₀ reduction of 501,187,233.627 CFUs would result in 50,118,723.3267 CFUs remaining.

TABLE 1

| Log₁₀ Reduction | Number of CFUs Remaining | Percent Reduction |
|---|---|---|
| 0 | 501,187,234 | 0% |
| 1 | 50,118,723 | 90% |
| 2 | 5,011,872 | 99% |
| 3 | 501,187 | 99.9% |
| 4 | 50,119 | 99.99% |
| 5 | 5,012 | 99.999% |
| 6 | 501 | 99.9999% |
| 7 | 50 | 99.99999% |
| 8 | 5 | 99.999999% |
| 9 | <1 | 99.9999999% |

When speaking in terms of bacterial, viral and fungal pathogens, a log₁₀ reduction is appropriate, as microorganisms are numbered in the millions or more due to the rapid doubling time thereof. As demonstrated herein, the log₁₀ reduction of PA disinfectant/sanitizer use solutions comprising concentrated PA disinfectant/sanitizer solutions diluted with water is significantly increased when mixed with a peracid booster (“PA booster”) as disclosed herein. For example, based on the MBEC data set forth below, efficacy of PA disinfectant/sanitizer use solutions can be increased by up to about 4 orders of magnitude.

It is believed that known use solutions made from concentrated PA disinfectants/sanitizers may not be effective at killing viruses, fungi and/or spores, much less penetrating, killing and/or removing biofilms. It has surprisingly been found that the efficacy of use solutions comprising concentrated PA disinfectants/sanitizers can be increased or “boosted” by mixing together a PAA boosting composition as described herein (hereinafter, “PA booster” or “PA boosters”) together with a PA disinfectant/sanitizer (and optionally, water), to make a use solution with improved properties. In particular, the PA disinfectant/sanitizer combined with a PA booster is not only effective at killing viruses, fungi and/or spores that are present on hard surfaces, but the boosted efficacy is possible without increasing the concentration of PAA in the resulting use solution and/or the PA disinfectant/sanitizer concentrate from which it is made. It has further surprisingly been found that by mixing together a PA booster with a PA disinfectant/sanitizer, smaller quantities of the PA disinfectants/sanitizers can be used to effectively treat hard surfaces and/or the amount of PA in the PA disinfectant/sanitizer concentrates can be reduced while still providing for use solutions having efficacy against microorganisms that would not otherwise be met. Moreover, the efficacy of PA in PA solutions can be increased by adding exemplary PA boosters thereto, such that the resulting use solutions may not only disinfect and/or sanitize hard surfaces, but can also provide for good biofilm penetration, kill and removal. These and other aspects of the disclosed invention are described herein below.

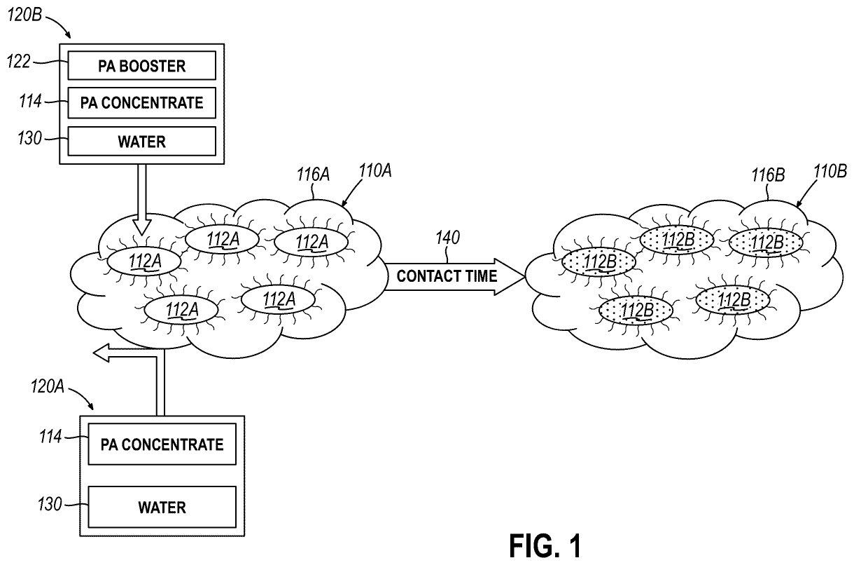

Referencing FIG. 1, a biofilm (110A), which is located on a hard surface (not shown) comprises live bacteria (112A) surrounded at least in part by an EPS (116A). A PA use solution (120A) made from a PA disinfectant/sanitizer concentrate (114) containing about 800 ppm of PAA and diluted with water (130) is applied to the biofilm (110A) for a contact time of up to 5 minutes. As can be seen in FIG. 1, the PA use solution (120A) does not substantially penetrate the EPS (116A) of the biofilm (110A).

Further referencing FIG. 1, an exemplary PA booster (122) is provided. As can be seen in FIG. 1, the PA booster (122) is mixed with the PA disinfectant/sanitizer concentrate (114) and water (130) to form a second PA use solution (120B) that at least initially contains the same total amount of PA that was originally present in the first PA use solution (120A), and that comprises only the PA disinfectant/sanitizer concentrate (114) and water (130).

The second PA use solution containing a PA booster (120B) is applied to the biofilm (110A) for a contact time of at least about 5 minutes, which is represented by the arrow (140) in FIG. 1. As can be seen on the right side of the arrow (140) in FIG. 1, the second PA use solution (120B) containing the PA booster penetrates the EPS (116B), so that PA in the PA use solution (120B) can reach and kill bacteria (112B) inside of the biofilm (110B) that has been breached.

Thus, referring to FIG. 1, it is believed that even though the same total amount of PAA is at least initially present in both of the PA use solutions (120A, 120B), and even though the concentration of PA is relatively reduced in the second PA use solution (120B) as compared to the first PA use solution (120A) due to the dilution by PA booster, the efficacy of the PA in the second PA use solution (120B) is “boosted” so that it not only effectively penetrate the EPS (116B), but it kills bacteria (112B) inside of the breached biofilm (110B), and the biofilm can then be removed.

Without wishing to be bound by theory, it is believed that the presence of a PA booster in the disclosed use solutions made with concentrated PA disinfectant/sanitizers allows for penetration of a biofilm that may not otherwise occur using known use PA disinfectant/sanitizer solutions, particularly without adding significant more PA thereto. It is further believed that the combination of short chain organic acid(s) having at least one pKa of from about 2 to about 7 with exemplary biodispersants in acidic PA boosters has a synergistic effect, such that a PA use solution as described herein can breach the protective EPS of a biofilm. It is further believed that the breach of the EPS of a biofilm allows PAA and/or and other antimicrobial components that are present in the disclosed use solutions may cross through the EPS and kill microbes contained therein, as compared to solutions such as those described in, for example, U.S. '789, which are not believed to be capable of penetrating a biofilm, much less within a contact time of 5 minutes or less.

Accordingly, the disclosed PA boosters provide what is believed to be heretofore undiscovered synergies with existing PAA disinfectants/sanitizers such that they can also be used as a sterilant and/or used to kill and remove biofilms, without undesirably adding more PA thereto.

As described above and demonstrated herein, the efficacy of the PA (e.g., PAA) in PA disinfectants/sanitizers is surprisingly increased or “boosted” by addition of a PA booster, without having to otherwise add more PA to the PA use solutions. Moreover, PA (e.g., PAA) efficacy in PAA disinfectant/sanitizer use solutions may be boosted such that the contact time of the PA use solutions may be comparatively reduced as compared to the PA disinfectant/sanitizer concentrates alone or diluted with water. Surprisingly, these advantages may be obtained despite a reduction in the concentration of PA in the resulting PA use solutions when mixed with a PA booster. Accordingly, the combination of a PA booster with a PA disinfectant/sanitizer concentrate may be used to increase the killing capabilities of a PA use solution while avoiding the aforementioned undesirable consequences of attempting to boost PA efficacy by adding yet more PA to a PA solution. The disclosed PA boosters, including both foaming and non-foaming boosters, may be used to boost efficacy of a PA disinfectant/sanitizer concentrate and/or use solutions by from about 2 log₁₀ to about 5 log₁₀ cycles, from about 2.5 log₁₀ to about 4.5 log₁₀ cycles, or from about 3 log₁₀ to about 4 log₁₀ cycles, without adding more PA thereto.

These and other features of exemplary PA boosters, kits containing them and methods of using them are disclosed hereinbelow.

Exemplary PA Boosters

Exemplary PA boosters may comprise a combination of components selected from the following: (A) an organic acid; (B) a biodispersant; (C) a chelant: (D) a pH buffering agent; (E) a foaming agent; (F) a defoaming agent; (G) a coupling agent; and combinations thereof.

A) Organic Acids

In one aspect, the PA booster may have an acidic pH and may contain a combination of acids. The combination of acids may comprise a primary organic acid and an ancillary organic acid that is different from the primary organic acid. In one aspect, the main purpose of the primary organic acid may be to act as an antimicrobial agent.

Exemplary primary organic acids can have secondary properties that can provide exemplary PA boosters with additional desired characteristics, including but not limited to, an acidic pH. Exemplary primary organic acids that may be used may be selected from: glycolic acid benzoic acid, fumaric acid, caprylic acid, lactic acid, propionic acid, and combinations thereof.

Exemplary foaming PA boosters may comprise from about 0.1% to about 30%, from about 0.5% to about 20%, or from about 1.0% to about 15% of one or more primary organic acids. In one aspect, the PA booster is a low/no foaming PA booster, and may comprise from about 1.0% to about 15%, from about 5.0% to about 13.0%, or from about 7% to about 10%, of one or more primary organic acids.

In addition to containing one or more primary organic acid(s), the PA booster may further comprise an ancillary organic acid that is different from the primary organic acid. In one aspect, the primary purpose of the ancillary organic acid is to act as an acidifying agent of the PA booster. An ancillary organic acid may further aid a PA use solution in breaching an EPS of a biofilm, and/or may enhance the antimicrobial properties of the PA use solution.

In one aspect, ancillary organic acids may have a carbon chain length of C6 or less and at least one pKa of from about 2 to about 7. The ancillary organic acids may be selected from, for example, acetic acid, formic acid, maleic acid, tartaric acid, and combinations thereof.

In one aspect, the foaming PA boosters may comprise from about 0.1% to about 20%, from about 0.5 to about 18%, from about 1.0% to about 15%, or from about 5.0% to about 10% of one or more ancillary organic acids. In one aspect, the low/no foaming PA boosters may comprise from about 0.5% to about 30%, from about 2.0% to about 20%, or from about 7% to about 10% of one or more ancillary organic acids.

(B) Biodispersants

In one aspect, the disclosed PA boosters may comprise one or more biodispersants. Without wishing to be bound by theory, it is believed that when a PA use solution is made with a PA booster as described herein, biodispersant(s) contained therein aid in the breach of an EPS layer of a biofilm to which the PA use solution has been applied. It is further believed that the breach of the EPS layer of a biofilm allows for any antimicrobial agents contained in the PA use solution to penetrate the EPS layer, enter the biofilm and kill bacteria contained therein. It is further believed that biodispersants aid in the removal of treated biofilm from hard surfaces. Biodispersants that may be used with the disclosed compositions include, for example, alkyl sulfosuccinate, alkyl sulfoacetate, alkylamide hydrolysate, and combinations thereof.

In one aspect, the foaming PA boosters may comprise from about 0.1% to about 10%, from about 0.5% to about 8%, from about 1.0% to about 7%, or from about 4.0% to about 6.0% of one or more biodispersants. In one aspect, the low/no foaming PA boosters may comprise from about 1.0% to about 10%, from about 1.0% to about 7%, or from about 3% to about 4% of one or more biodispersants.

(C) Chelants

In one aspect, the PA boosters may contain one or more chelants. Without wishing to be bound by theory, it is believed that when a PA use solution is made with an exemplary PA booster as described herein, the chelant(s) contained therein may aid in the breach of an EPS layer of a biofilm to which the PA use solution has been applied by sequestering extracellular metals within the biofilm and thereby destabilizing biofilm cells.

Chelants of use in the disclosed PA boosters may include, for example, citric acid; 1-hydroxyethylidene 1,1-diphosphonic acid (“HEDP”); trisodium salt of methylglycinediacetic acid (for example, Trilon® M Liquid from BASF (Florham Park, NJ)); L-glutamic acid N,N-diacetic acid, tetrasodium salt (i.e., tetrasodium salt of GLDA; e.g., Biopure™ GLDA from Jarchem Innovative Ingredients LLC (Newark, NJ); hydroxyethylene-diaminetriacetic acid; trisodium salt (i.e., trisodium HEDTA; e.g. Dissolvene Na₃ from Nouryon (Chicago, IL); salts of ethylenediamine tetracetic acid (“EDTA”), and combinations thereof. Salts of EDTA may include tetrasodium salt of EDTA (e.g., Versene™ 100 from The Dow Chemical Company (Torrance, CA)).

In one aspect, the foaming PA booster may comprise from about 0.3% to about 20%, from about 0.5% to about 17%, from about 5.0% to about 18%, from about 8.0% to about 15%, or from about 9.0% to about 11%, of one or more chelants. In one aspect, the low/no foaming PA boosters may comprise from about 2.0% to about 20%, from about 6.0% to about 18%, or from about 9.0% to about 17% of one or more chelants.

(D) pH Buffering Agents

In one aspect, the PA Booster may have a pH of from about 3 to about 4, or from about 3.5 to about 4. To maintain their pH during storage, the PA boosters may contain one or more buffering agents. In one aspect, the pH buffering agents of use may also help to maintain the pH of use solutions comprising the PA boosters, concentrated PA disinfectants/sanitizers and water, such that the use solution has a pH of from about 1 to about 5.

Exemplary pH buffering agents may be chosen by one of ordinary skill in the art based upon the teachings herein. Suitable pH buffering agents include, for example, acetates, malates, fumarates, oxalates, and combinations thereof. Exemplary pH buffering agents include sodium acetate, sodium malate, sodium fumarate, sodium oxalate, and combinations thereof.

Exemplary foaming PA boosters and exemplary low/no foaming PA boosters may comprise from about 0.1% to about 5%, from about 0.15% to about 4%, from about 1% to about 3%, or from about 1.5% to about 2%, of one or more buffering agents.

(E) Foaming Agents

The PA booster may contain one or more foaming agents. Without wishing to be bound by theory, it is believed that the foaming agent may stabilize the bubble structure and/or matrix of compositions and/or prevent dissipation thereof after application to a hard surface.

Exemplary foaming agents may be selected from: ethoxylated alcohols, amine oxides, sulfonic acids, and combinations thereof. For example, foaming agents may be selected from: alcohol ethoxylate (C9-C11), myristyl amine oxide; cocoamine oxide, linear alkylbenzene sulfonate (e.g., DDBSA), and combinations thereof.

Exemplary foaming PA boosters may comprise from about 0.1% to about 15%, from about 0.7% to about 12%, or from about 1% to about 8%, or from about 2% to about 4%, of one or more foaming agents.

(F) Defoaming Agent

In one aspect, the PA booster may be a low/no foaming PA booster, and may comprise a defoaming agent (i.e., “defoamer”). Without wishing to be bound by theory, it is believed that the defoaming agents act to destabilize bubble structure and/or the bubble matrix so that the composition does not substantially foam, and if so, it dissipates quickly, when applied to a hard surface. Exemplary defoaming agents may include, for example, polyoxypropylene-polyethylene block copolymers, such as those available in the Pluronic® L series from BASF (Florham Park, NJ).

Exemplary low/no foaming PA boosters may comprise from about 4% to about 15%, from about 5% to about 9%, or from about 6% to about 8% of one or more defoaming agents.

(G) Coupling Agents

Low/no foaming PA boosters may contain one or more coupling agents. Without wishing to be bound by theory, it is believed that the coupling agents act to dissolve components in exemplary PAA boosters that would not otherwise dissolve therein. Exemplary coupling agents may include, for example, iminodipropate alanate, available as Amphoteric 400 from Evonik (Essen, Germany).

Exemplary low/no foaming PA boosters may comprise from about 4% to about 15%, from about 5% to about 9%, or from about 6% to about 8% of one or more defoaming agents.

Preparation of Boosted PA Disinfectant/Sanitizer Use Solutions

As demonstrated herein below, the log₁₀ reduction of PA disinfectant/sanitizer use solutions comprising concentrated PA disinfectant/sanitizer solutions diluted with water, is significantly increased when instead diluted with exemplary PA boosters and water. For example, an exemplary PA booster may be added to a concentrated PA disinfectant/sanitizer and the resulting mixture may be diluted with water to form the PA disinfectant/sanitizer use solution; Formula I below shows how such a dilution may be set forth:

Xoz:Yoz:Zoz=concentrated PA disinfectant/sanitizer:PA booster:diluent

Biofilm (Foaming Booster)

For example, a foaming PAA disinfectant/sanitizer use solution may comprise: from about 1 oz to about 3 oz, or about 2 oz concentrated PAA disinfectant/sanitizer (containing between 5% and 6% of active PAA):about 8 oz to about 15 oz, or about 12 oz to about 14 oz, or about 13 oz of Foaming PAA Booster:about 100 oz to about 125 oz, or 113.2 oz of water.

In another aspect, a PAA disinfectant/sanitizer (containing between 5% and 6% active PAA) use solutions of PAA/Foaming PAA Booster can be made with concentrations of PAA between 200 ppm to 850 ppm PAA and 12.8 oz to 16.0 oz Foaming PAA booster. Examples of PAA disinfectant/sanitizer:PAA foaming booster:diluent concentrations are:

0.5 oz PAA: 12.8 oz Foaming Booster: 114.7 oz water to 2.1 oz PAA: 16.0 oz Foaming Booster: 113.1 oz water or various concentrations of each constituent between the ranges the with water to have a final volume of 128 oz.

5 mL PAA: 30 mL Foaming Booster: 965 mL water to 17 mL PAA: 100 mL Foaming Booster: 883 mL water or various concentrations of each constituent between the ranges with water to have a final volume of 1 L.

Biofilm (no/low foam booster)

In a further example, a low/no foaming PAA disinfectant/sanitizer use solution is made by blending together: 2.5 oz concentrated PAA disinfectant/sanitizer (containing between 5% and 6% of active PAA):0.5 oz of low/no foam PAA booster:125.0 oz of water.

In another aspect, a PAA disinfectant/sanitizer (containing between 5% and 6% active PAA) use solutions of PAA/low/no foam PAA booster can be made with concentrations of PAA between 200 ppm to 850 ppm PAA and 0.5 oz to 2.0 oz of low/no foam PAA booster. Examples of PAA disinfectant/sanitizer:PAA foaming booster:low/no foam PAA booster:diluent concentrations are:

0.5 PAA: 0.5 oz no/low foam PAA Booster: 127.0 oz water to 2.1 oz PAA: 2.0 oz Foaming Booster: 125.5 oz water or various concentrations of each constituent between the ranges the with water to have a final volume of 128 oz.

10 mL PAA: 3 mL no/low foam PAA Booster: 970 mL water to 17 mL PAA: 5 mL Foaming Booster: 978 mL water or various concentrations of each constituent between the ranges with water to have a final volume of 1 L.

Sporicidal (Foaming Booster)

In another aspect, a PAA disinfectant/sanitizer (containing between 5% and 6% active PAA) use solutions of PAA/Foaming PAA Booster can be made with concentrations of PAA between 500 ppm to 850 ppm PAA and 12.8 oz to 16.0 oz Foaming PAA booster. Examples of PAA disinfectant/sanitizer:PAA foaming booster:diluent concentrations are:

1.2 oz PAA: 12.8 oz Foaming Booster: 114.7 oz water to 2.1 oz PAA: 16.0 oz Foaming Booster: 113.1 oz water or various concentrations of each constituent between the ranges the with water to have a final volume of 128 oz.

5 mL PAA: 30 mL Foaming Booster: 965 mL water to 17 mL PAA: 100 mL Foaming Booster: 883 mL water or various concentrations of each constituent between the ranges with water to have a final volume of 1 L.

Sporicidal (no/low foam booster)

In another aspect, a PAA disinfectant/sanitizer (containing between 5% and 6% active PAA) use solutions of PAA/Foaming PAA Booster can be made with concentrations of PAA between 500 ppm to 850 ppm PAA and 12.8 oz to 16.0 oz Foaming PAA booster. Examples of PAA disinfectant/sanitizer:PAA foaming booster:diluent concentrations are:

1.2 oz PAA: 0.5 oz no/low foaming Booster: 126.3 oz water to 2.1 oz PAA: 16.0 oz low/no Foaming Booster: 123.9 oz water or various concentrations of each constituent between the ranges the with water to have a final volume of 128 oz.

10 mL PAA: 3 mL Foaming Booster: 987 mL water to 17 mL PAA: 5 mL Foaming Booster: 978 mL water or various concentrations of each constituent between the ranges with water to have a final volume of 1 L.

To calculate the amount of ppm of PAA in the resulting foaming and low/no foaming disinfectant/sanitizer use solutions, the following Formula II may be used:

$$ppm PAA = ( \frac{\begin{matrix}oz of PAA concentrate \times \% active \\ PAA in concentrate\end{matrix}}{Total oz} ) \times 10000$$

Using formula II, if a concentrated PAA disinfectant/sanitizer containing 5% active PAA is combined with an exemplary foaming or low/no foaming PA booster and diluent as described above, the total amount of PAA in the respective foaming or low/no foaming PAA disinfectant/sanitizer use solution would contain 781.25 ppm of active PAA.

To calculate the amount of ppm of active organic acid in use solution, the following Formula III may be used:

$$ppm Organic Acid = ( \frac{\begin{matrix}oz of Booster product \times \% active \\ Organic Acid in Booster Product\end{matrix}}{Total oz} ) \times 10000$$

For example, if a foaming PA booster containing 1% of an active organic acid was used, the total ppm of the active organic acid in the use solution is equal to 1,000 ppm of active organic acid.

Kits

Exemplary PA boosters may be packaged and sold together with a concentrated PA disinfectant/sanitizer. In exemplary kits, the PA booster may be packaged in a first container (e.g., a first bottle) and the concentrated PA disinfectant/sanitizer may be packaged in a second container (e.g., a second bottle). Exemplary concentrated PA disinfectants/sanitizers may contain a PA selected from: peroxyacetic acid, peroxyoctanoic acid, peroxysulfonic acid, peroxyformic acid, and combinations thereof. Exemplary concentrated PA disinfectants/sanitizers may contain a total of from about 2% to about 15%, from about 3% to about 10%, or from about 4% to about 6% of one or more peracids. Exemplary concentrated PA disinfectants/sanitizers may have a pH of from about 1 to about 5, or from about 1 to about 4.

Method of Use

The disclosed compositions may be used for reducing and/or eliminating and/or substantially eliminating a biofilm on a surface, such as a device surface, or a surface or device surface associated with a health care or veterinary environments (e.g., a surgery/operation room). Such surfaces may be selected from a stainless-steel surface, a glass surface, a PVC surface such as a wall sheeting. In one aspect, the surface may be a floor, for example, a floor surface comprising sheet vinyl, sheet rubber, urethane-coated epoxy, urethane-coated linoleum (poured flooring). In one aspect, the surface may be a medical device, for example a medical device comprising stainless steel, or chrome-plating over steel.

In one aspect, a method for disinfecting, sterilizing, and/or sanitizing a surface intended for human or veterinary medical use is disclosed. In this aspect, the method may comprise contacting the surface with mixture comprising a) a peracid-containing composition and b) a peracid (PA) boosting composition as disclosed herein. For example, the PA boosting composition may comprise a primary acid, an optional ancillary acid, a biodispersant, a chelant, a pH buffering agent, a foaming agent, a defoaming agent, and a coupling agent, as described herein.

In one aspect, the surface may be a surface of a hospital room, for example, one or more surfaces being selected from a floor, a wall, a ceiling, a window, a table, a sink, a bedrail, a counter, medical equipment, and combinations thereof. In one aspect, the surface may be an operating room. In a further aspect, the surface may be a medical device, for example a medical device that is designed to come in contact with a patient (human or animal) for example a surgical device, endoscope, or the like.

In one aspect, a method for disinfecting and/or sanitizing a surface intended for manufacture of a cosmetic is disclosed. Such cosmetics may include, for example a skin care product, a hair care product, a body care product, or the like. The method may comprise contacting the surface with a peracid-containing composition and a peracetic acid (PAA) boosting composition as described herein. Exemplary surfaces include, but are not limited to a surface of a manufacturing plant floor, ceiling, manufacturing equipment, packaging area, receiving area, storage area for raw materials, storage area for finished goods, and combinations thereof.

In one aspect, a method for disinfecting and/or sanitizing on a surface intended for manufacture of a pharmaceutical is disclosed. In this aspect, the method may comprise contacting the surface with a peracid-containing composition and a peracetic acid (PAA) boosting composition as disclosed herein. In one aspect, the surface may be a surface of a manufacturing plant floor, ceiling, manufacturing equipment, packaging area, receiving area, storage area for raw materials, storage area for finished goods, and combinations thereof.

In one aspect, the disinfecting, sterilizing, and/or sanitizing may achieve one or more of reducing, deactivating, removing, and/preventing a virus, an endospore, a bacterium, and combinations thereof. Such disinfecting, sterilizing, and/or sanitizing may result in a 100% destruction of said microorganism, or at least 99.9%, or at least 99.8%, or at least 99.7%, or at least 99.6%, or at least 99.5%, or at least 99.4%, or at least 99.3%, or at least 99.2%, or at least 99.1%, or at least 99%, or at least 98%, or at least 97%, or at least 96%, or at least 95% destruction of said microorganism. In a further aspect, the disinfecting, sterilizing, and/or sanitizing reduces, deactivates, removes, and/or prevents a biofilm. The biofilm may contain one or more of a bacterium, a virus, an endospore, a bacterium, or combinations thereof. In one aspect, the microorganism or biofilm may comprise an organism according to Table 8.

In one aspect, the mixture used in the methods may comprise from about 10 ppm to 1000 ppm peracid, or from about 20 ppm to about 900 ppm peracid, or from about 30 ppm to about 800 ppm peracid, or from about 30 ppm to about 700 ppm peracid, or from about 40 ppm to about 600 ppm peracid, or from about 50 ppm to about 500 ppm peracid, or from about 60 ppm to about 400 ppm peracid, or from about 70 ppm to about 300 ppm peracid, or from about 80 ppm to about 200 ppm peracid, or from about 90 ppm to about 100 ppm peracid. In one aspect, the peracid may be peracetic acid. In a further aspect, the mixture may comprise at least 300 ppm of said primary acid, or from about 300 to about 600 ppm of said primary acid, or from about 400 to about 500 ppm of said primary acid. In a yet further aspect, the mixture may comprise at least 500 ppm of said primary acid and ancillary acid combined, or from about 500 to about 1500 ppm of said primary acid and ancillary acid combined, or from about 600 to about 1200 ppm of said primary acid and ancillary acid combined. As set forth above, the mixtures may comprise a primary acid that is different from the optional ancillary acid. In certain aspects, the primary and/or ancillary acid may provides\ an acid pH to said mixture and/or said PA boosting composition.

The PA boosting composition used may be selected based on the type of use of the mixture. For example, in one aspect, the PA boosting composition may be a low/no foaming composition, which may be useful for CIP indications. In a further aspect, the PA boosting composition may be a foaming composition. Such foaming compositions may be useful for an environmental application or a COP application.

The methods may employ a contacting step of various lengths of time. For example, the contacting may be for a period of time of from about 0.5 to about 60 minutes, or from about 0.5 to about 10 minutes, or about 2 to about 8 minutes, or about 4 to about 8 minutes, or about 6 minutes. Likewise, the contacting may be carried out a various temperatures. Such temperature may include a range of from about 15° C. to about 60° C., or from about 18° C. to about 60° C., or from about 18° C. to about 56° C., or from about 18° C. to about 50° C., or from about 18° C. to about 40° C., or from about 18° C. to about 30° C., or from about 18° C. to about 24° C.

An exemplary method of boosting efficacy of a PA disinfectant/sanitizer use solution may comprise the following steps. A concentrated PA disinfectant/sanitizer is diluted with either a foaming PA booster or a low/no foaming PA booster and water. For example, a concentrated PA disinfectant/sanitizer comprising one or more peracetic acids at from about 2% to about 15% by weight is diluted with water to produce a first use solution having (a) a pH of from about 1 to about 5; (b) less than or equal to about 800 ppm total peroxygen species; and (c) is characterized by an efficacy of equal to or less than a 5 log₁₀ reduction of a Pseudomonas aeruginosa biofilm after contact therewith for 10 minutes or more pursuant to ASTM E2871-12 (approved Apr. 1, 2012 & published in June of 2012).

The same concentrated PA disinfectant/sanitizer may instead diluted as follows:

A foaming PAA disinfectant/sanitizer use solution is made by blending together: 2 oz concentrated PAA disinfectant/sanitizer (containing between 5% and 6% of active PAA):12.8 oz of Foaming PAA Booster:113.2 oz of water. A low/no foaming PAA disinfectant/sanitizer use solution is made by blending together: 2.5 oz concentrated PAA disinfectant/sanitizer (containing between 5% and 6% of active PAA):0.5 oz of low/no foam PAA booster:125.0 oz of water. The second use solution: (a) has a pH of from about 1 to about 5; and (b) is characterized by a boosted efficacy equal to or greater than 6 log₁₀ reduction of Pseudomonas aeruginosa biofilm after contact Therewith for 10 minutes or more pursuant to ASTM E2871-12 (approved Apr. 1, 2012 & published in June of 2012).

The above-described compositions may then be applied to a surface, which may include applying via a COP application, a CIP application, or an environmental application. Typically, a low or no-foaming formulation will be used for a CIP application, whereas a foaming formulation will be used for a COP or environmental application. The application may be rinsed following contact, such contact being of a time sufficient to achieve the desired result.

Examples and Data

Exemplary foaming PA boosters A through O are set forth in Table 2, and exemplary low/no foaming PA boosters I through XIV are set forth in Table 3 below. It is to be noted that the weight percentages of components set forth in Tables 2 and 3 are active weight percentages.

TABLE 2

| Chemical Name | A | B | C | D | E | F | G | H | I | J | K | L | M | N | O |
|---|---|---|---|---|---|---|---|---|---|---|---|---|---|---|---|
| Ancillary Acid | | | | | | | | | | | | | | | |
| acetic acid | 0.5 | 5 | | | 5 | 10 | 15 | 20 | 5 | 0.5 | 0.5 | 0.5 | 0.5 | 0.5 | 0.5 |
| formic acid | | | | | 1 | | | | 1 | | | | | | |
| maleic acid | | | 7.5 | | | | | | | | | | | | |
| tartaric acid | | | | 5 | | | | | | | | | | | |
| Primary Acid | | | | | | | | | | | | | | | |
| glycolic acid | 0.6 | 5 | 20 | 7.5 | 15 | 5 | 10 | 0.6 | 0.6 | 4 | 5 | 3 | 5 | 5 | |
| benzoic acid | | | | | | | | | | 10 | | | | | |
| fumaric acid | | | | | | | | | | | 5 | | | | |
| caprylic acid | | | | | | | | | | | | 5 | | | |
| lactic acid | | | | | | | | | | | | | S | | 0.6 |
| proprionic acid | | | | | | | | | | | | | | 2 | |
| Bio-dispersant | | | | | | | | | | | | | | | |
| sodium dioctyl | 0.4 | 4 | 5 | | 4 | 1 | 10 | | | 4 | 4 | 4 | | 6 | 0.4 |
| sulfosuccinate | | | | | | | | | | | | | | | |
| disodium lauryl | | | | 4 | | | | 3 | | | | | | | |
| sulfosuccinate | | | | | | | | | | | | | | | |
| sodium lauryl | | | | | | | | | 5 | | | | 6 | | |
| sulfoacetate | | | | | | | | | | | | | | | |
| Chelant | | | | | | | | | | | | | | | |
| citric acid | 0.4 | 4 | 4 | | | 8 | 5 | | | 4 | 4 | 4 | 4 | 5 | 0.4 |
| 1-hydroxyethylidene | 0.3 | 6 | 8 | | 5 | 10 | 9 | 3 | 4 | 6 | 5 | 8 | 10 | 0.3 | 0.3 |
| 1,1-diphosphonic | | | | | | | | | | | | | | | |
| acid | | | | | | | | | | | | | | | |
| methylglycinediacetic | | | | | | | 0.75 | | | | | | | | |
| acid | | | | | | | | | | | | | | | |
| L-glutamic acid N,N- | | | | | | 0.5 | | | | | | | | | |
| diacetic acid, | | | | | | | | | | | | | | | |
| tetrasodium salt (i.e., | | | | | | | | | | | | | | | |
| tetrasodium salt of | | | | | | | | | | | | | | | |
| GLDA) | | | | | | | | | | | | | | | |
| hydroxyethylene- | | | | | | | | 8 | | | | | | | |
| diaminetriacetic acid | | | | | | | | | | | | | | | |
| trisodium salt (i.e., | | | | | | | | | 5 | | | | | | |
| trisodium HEDTA) | | | | | | | | | | | | | | | |
| salts of | | | | | | | | | | | | | | | |
| ethylenediamine | | | | | | | | | | | | | | | |
| tetracetic acid | | | | | | | | | | | | | | | |
| including, but not | | | | | | | | | | | | | | | |
| limited to, sodium | | | | | | | | | | | | | | | |
| salts of EDTA (e.g., | | | | | | | | | | | | | | | |
| tetrasodium salt of | | | | | | | | | | | | | | | |
| EDTA) | | | | | | | | | | | | | | | |
| ethylenediamine | | | | | 3 | | | | | | | | | | |
| tetracetic acid | | | | | | | | | | | | | | | |
| Foaming Agent | | | | | | | | | | | | | | | |
| alcohol ethoxylate | 0.3 | 3 | | | 0.3 | 0.3 | | 3 | 1 | 2 | 8 | 3 | 4 | 3 | 0.3 |
| (C9-C11) | | | | | | | | | | | | | | | |
| myristyl amine oxide | | | | | | | | | | | | | | | |
| Cocoamine oxide | | | 5 | | | | | | | | | | | | |
| DDBSA (Linear | 0.4 | 4 | 8 | | 0.4 | 0.4 | 6 | 5 | 6 | 3 | 4 | 4 | 4 | 4 | 0.4 |
| Alkylbenzene | | | | | | | | | | | | | | | |
| sulfonate) | | | | | | | | | | | | | | | |
| pH Buffering Agent | | | | | | | | | | | | | | | |
| sodium acetate | 0.15 | 1.5 | | 3 | | 3 | 4 | 3 | 2 | 3 | 1 | 2 | 4 | 2 | 0.15 |
| Sodium Malate | | | 4 | | | | | | | | | | | | |
| Sodium Fumarate | | | | | 4 | 0.5 | | | | | | | | | |
| Sodium Oxalate | | | | | | | | | 3 | | | | | | |

QS with Water to 100

TABLE 3

| | I | II | III | IV | V | VI | VII | VIII | IX | X | XI | XII | XIII | XIV |
|---|---|---|---|---|---|---|---|---|---|---|---|---|---|---|
| Ancillary Acid | | | | | | | | | | | | | | |
| acetic acid | 2.5 | 2.5 | 2.5 | 2.5 | | 7.5 | 8 | 10 | 5 | 2.5 | 5 | 10 | 20 | 30 |
| formic acid | | | | | | | | | 3 | | 3 | | | |
| maleic acid | | | | | | | | | | 5 | | | | |
| tartaric acid | | | | | | | | | | | | 3 | | |
| Primary Acid | | | | | | | | | | | | | | |
| glycolic acid | 10 | 10 | 10 | 10 | 10 | 10 | 10 | 10 | 10 | 10 | 15 | 10 | 10 | 12.5 |
| benzoic acid | | | | | 10 | | | | | | | | | |
| fumaric acid | | | | | | 10 | | | | | | | | |
| caprylic acid | | | | | | | 10 | | | | | | | |
| lactic acid | | | | | | | | 12 | | | | | | |
| proprionic acid | | | | | | | | | 10 | | | | | |
| Bio-dispersant | | | | | | | | | | | | | | |
| sodium dioctyl | 3 | 3 | 3 | 3 | 4 | | | 4 | | | 6 | 3 | 10 | 4 |
| sulfosuccinate | | | | | | | | | | | | | | |
| disodium lauryl | | | | | | 3 | | | 4 | | | | | |
| sulfosuccinate | | | | | | | | | | | | | | |
| sodium lauryl | | | | | | | 3 | | | 4 | | | | |
| sulfoacetate | | | | | | | | | | | | | | |
| Chelant | | | | | | | | | | | | | | |
| citric acid | 4 | 4 | 4 | 4 | 10 | 4 | 4 | 4 | 4 | | | | | 4 |
| 1-hydroxyethylidene | 2.4 | 2.4 | 2.4 | 2.4 | 10 | 5 | 4 | 6 | 6 | 9 | 9 | 2.4 | 2.4 | 6 |
| 1,1-diphosphonic acid | | | | | | | | | | | | | | |
| methylglycinediacetic | | | | | | | | | | 8 | | | | |
| acid | | | | | | | | | | | | | | |
| L-glutamic acid N,N- | | | | | | | | | | | 4 | | | |
| diacetic acid, | | | | | | | | | | | | | | |
| tetrasodium salt (i.e., | | | | | | | | | | | | | | |
| tetrasodium salt of | | | | | | | | | | | | | | |
| GLDA) | | | | | | | | | | | | | | |
| hydroxyethylene- | | | | | | | | | | | | | | |
| diaminetriacetic acid | | | | | | | | | | | | | | |
| trisodium salt (i.e., | | | | | | | | | | | | 3 | | |
| trisodium HEDTA) | | | | | | | | | | | | | | |
| salts of | | | | | | | | | | | | | | |
| ethylenediamine | | | | | | | | | | | | | | |
| tetracetic acid | | | | | | | | | | | | | | |
| including, but not | | | | | | | | | | | | | | |
| limited to, sodium | | | | | | | | | | | | | | |
| salts of EDTA (e.g., | | | | | | | | | | | | | | |
| tetrasodium salt of | | | | | | | | | | | | | | |
| EDTA) | | | | | | | | | | | | | | |
| ethylenediamine | | | | | | | | | | | | | 4 | |
| tetracetic acid | | | | | | | | | | | | | | |
| Coupling Agent | | | | | | | | | | | | | | |
| iminodipropate | 3.6 | 2 | 3 | 4 | 3.6 | 5 | 3.6 | 3.6 | 3.6 | 3.6 | 3.6 | 3.6 | 10 | 8 |
| alanate | | | | | | | | | | | | | | |
| Defoamer | | | | | | | | | | | | | | |
| polyoxypropylene- | 8 | 10 | 8 | 10 | 15 | 6 | 7 | 9 | 8 | 8 | 8 | 8 | 8 | 10 |
| polyethylene block | | | | | | | | | | | | | | |
| copolymer | | | | | | | | | | | | | | |
| pH Buffering Agent | | | | | | | | | | | | | | |
| sodium acetate | 1.5 | 1.5 | 1.5 | 1.5 | 5 | 1.5 | 3 | 1.5 | 5 | 2 | 3 | 3 | 3 | 5 |
| Sodium Malate | | | | | | | | | | | | | | |
| Sodium Fumarate | | | | | | | | | | | | | | |
| Sodium Oxalate | | | | | | | | | | 3 | | | | |

QS with Water to 100

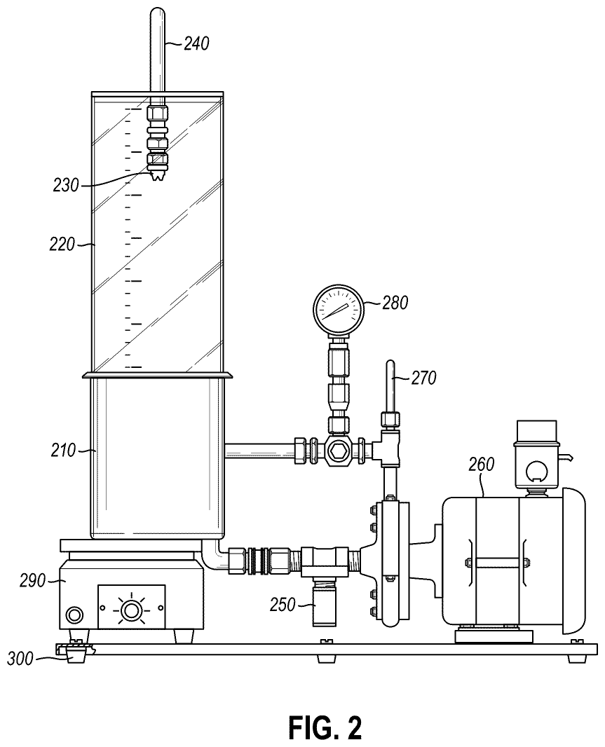

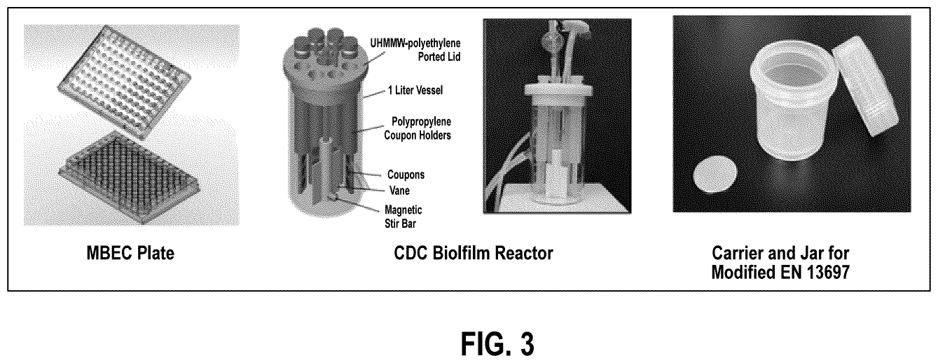

Foaming characteristics of exemplary PA boosters and/or PA use solutions comprising them are determined using a recirculating foam tester (“RALF”), which is shown in FIGS. 2 and 3. A RALF (200) comprises the following components: a stainless steel beaker that is 6 inches in diameter by 6 inches tall (210); a polycarbonate cylinder having height markings that is 6 inches in diameter by 18 inches tall (220) that is sealed to the stainless steel beaker (210); a VeeJet® low pressure 20 degree tapered-edge, wide tip spray nozzle (230) from Spraying Systems Co.® that is connected to a screw valve (232) to regulate flow and pressure through the valve; ⅜ inch stainless steel piping comprising feed and return lines (240) that draws solution from the bottom of the stainless steel beaker (210) and sprays the solution back into the beaker through the spray nozzle (230); a gate valve to drain system (250); a ⅕ horsepower electric recirculating pump (260); a thermometer or thermocouple port (270); a pressure gauge (280); a hot plate (290); a stainless steel base with isolator feet (300).

Using a RALF, foaming characteristics of a solution are determined as follows. 3.0 L of DI water is placed inside of the stainless-steel beaker (tap water or hard water may be used depending on test parameters). A recirculating pump is used to recirculate the water until the desired testing temperature is reached (temperature is measured using a thermometer inserted into the thermometer/thermocouple port. If testing at room temperature, leave the hot plate off, otherwise, heat the water to about 35° C.-60° C. Once the water reaches the desired temperature, dose in the correct amount of test solution. Operate the RALF at a pressure of 40 psi and a flow rate of 2 gallons/minute for 10 minutes, allowing any foam to build. During testing, record the foam height of the test solution at various times. If foam height is uneven, take the average of the high side of the foam and the low side of the foam. Turn the RALF off to stop the flow. Record the time it takes for the foam to break to a height of 0 inches.

Foaming characteristics of foaming PA booster example A and no/low foaming PA boosters in accordance with examples I through IV are measured and set forth in Table 4 below:

TABLE 4

| | | | |
|---|---|---|---|
| | Temp | | |
| Time (min) | (° C.) | Height (in) | Dissipation Time (min) |

Example A

| | | | |
|---|---|---|---|
| 0.25 | 20 | 18 | 120 |
| 0.25 | 20 | 18 | 140 |
| 0.25 | 20 | 18 | 120 |

Example I

| | | | |
|---|---|---|---|
| 1 | 35 | 1 | 0.15 |
| 5 | 35 | 2 | 0.5 |
| 10 | 35 | 4 | 0.75 |

Example II

| | | | |
|---|---|---|---|
| 1 | 35 | 1.5 | 0.25 |
| 5 | 35 | 2.5 | 0.8 |
| 10 | 35 | 4.5 | 1.15 1 |

Example III

| | | | |
|---|---|---|---|
| 1 | 35 | 0.5 | 0.2 |
| 5 | 35 | 1.25 | 1 |
| 10 | 35 | 4 | 2.25 |

Example IV

| | | | |
|---|---|---|---|
| 1 | 35 | 0.5 | 0.3 |
| 5 | 35 | 1.25 | 1 |
| 10 | 35 | 3.5 | 1.5 |

Referencing Table 5, boosting efficacy of an exemplary foaming PA booster in accordance with Example A is assessed using the “Standard Test Method for Testing Disinfectant Efficacy against Pseudomonas aeruginosa Biofilm using the MBEC Assay” (ASTM E2799-17 (approved Apr. 1, 2017 & published in May of 2017); hereinafter, “MBEC”). Proxitane® EQ Peracetic Acid from Solvay Chemicals, Inc. (Houston, TX) is chosen as an exemplary commercially available PAA to be mixed with the exemplary PA boosters in accordance with the present disclosure. Proxitane® EQ Peracetic Acid is an EPA registered liquid product that is marketed for circulation cleaning and institutional/industrial sanitizing of pre-cleaned, hard, non-porous food contact surfaces and equipment such as tanks, pipelines, evaporators, fillers, pasteurizer and aseptic equipment. According to its MSDS, Proxitane® EQ Peracetic Acid contains: 5-5.4 wt % of peracetic acid; 20-24 wt % of hydrogen peroxide; 10-12 wt % of acetic acid; and the balance is water.

TABLE 5
Reduction of P. aeurginosa biofilm (log CFU) upon exposure to
PAA in the absence or presence of Booster

| | Booster Presence¹ | | |
|---|---|---|---|
| | | Low/No Foam | High Foam |
| PAA (ppm) | None | (Example I) | (Example A) |
| 0 | NA² | 2.66 | 5.37 |
| 10 | NR³ | ND⁴ | 3.19 |
| 25 | NR | 4.70 | ND |
| 50 | NR | >7.66 | >7.66 |
| 100 | 2.66 | >7.66 | >7.66 |
| 200 | 5.52 | >7.66 | >7.66 |
| 300 | 4.01 | >7.66 | >7.66 |
| 400 | 2.66 | >7.66 | >7.66 |

¹At 0.5 oz/gal low/no foam Booster or 12.8 oz/gal high foam Booster.
²Not applicable. Initial cell density was 8.66 log CFU.
³No reduction.
⁴Not determined.

As can be seen from the MBEC data set forth in Table 5, a PA use solution comprising Proxitane® EQ Peracetic Acid diluted with water results in an efficacy characterized by a log₁₀ reduction of 2.66 or 5.52, even when the PAA concentration in the PA use solution is as high as 400 ppm. In contrast, when a PA use solution comprising an exemplary foaming booster, Proxitane® EQ Peracetic Acid and water is tested, an efficacy characterized by a log₁₀ reduction of 7.7 is achieved, even when the PAA concentration in the PA use solution is as low as 50 ppm. Thus, it can be concluded that with respect to Proxitane® EQ Peracetic Acid, an exemplary PA booster “boosts” the efficacy of Proxitane® EQ Peracetic Acid from about 4 to about 5 log₁₀ cycles without the addition of more PAA to the use solutions.

Referencing Table 6, boosting efficacy of an exemplary foaming PA booster in accordance with Example A may be further assessed using the “Standard Test Method for Testing Disinfectant Efficacy against Pseudomonas aeruginosa Biofilm using the MBEC Assay” (ASTM E2871-19 (approved Jan. 1, 2019 & published in February of 2019). Proxitane® EQ Peracetic Acid from Solvay Chemicals, Inc. (Houston, TX) is once again chosen as an exemplary commercially available PAA to be mixed with the exemplary PA boosters in accordance with the present disclosure.

TABLE 6
Reduction of P. aeurginosa biofilm (log CFU) upon exposure to PAA in the absence or presence of
Booster by ASTM E2871-19

| | | Booster Presence² | | Efficacy Increase³ | |
|---|---|---|---|---|---|
| | PAA¹ Initial | PAA¹ + Low/No | PAA¹ + High | PAA¹ + Low/No | PAA¹ + High |
| Test | Efficacy | Foam (Example I) | Foam (Ex. A) | Foam (Ex. I) | Foam (Ex. A) |
| 1 | 2.99 | 7.98 | 8.26 | 4.99 | 5.27 |
| 2 | 3.10 | 6.39 | 7.60 | 3.29 | 4.50 |
| 3 | 3.00 | 6.20 | 7.48 | 3.20 | 4.48 |
| 4 | 3.97 | 8.24 | 8.21 | 4.27 | 4.24 |

¹PAA concentration 800 ppm
²At 0.5 oz/gal low/no foam Booster or 12.8 oz/gal high foam Booster.
³Log CFU

As can be seen in Table 6, a PA use solution comprising Proxitane EQ Peracetic Acid and water, in which PAA is present at a concentration of 800 ppm, is characterized by an average efficacy of a 3.27 log₁₀ reduction of Pseudomonas aeruginosa biofilm after contact therewith for 10 minutes or more pursuant to ASTM E2871-19. Moreover, a PA use solution comprising an exemplary no/low foaming PA Booster in addition to Proxitane® EQ Peracetic Acid and water, is characterized by an average efficacy of a 3.97 log₁₀ reduction of Pseudomonas aeruginosa in the biofilm after contact therewith for 10 minutes or more pursuant to ASTM E2871-19. Moreover, a PA use solution comprising an exemplary foaming PA Booster in addition to Proxitane® EQ Peracetic Acid and water, is characterized by an average efficacy of a 4.6 log₁₀ reduction of Pseudomonas aeruginosa biofilm after contact therewith for 10 minutes or more pursuant to ASTM E2871-19. Thus, it can be concluded that exemplary PA boosters “boost” the efficacy of Proxitane® EQ Peracetic Acid from about 2 to 5 log₁₀ cycles without the addition of more PAA to the use solutions.

Any impact of the primary acid that is present in exemplary PA boosters may be assessed via MBEC. Referencing Table 7, data relating to the reduction of P. aeruginosa biofilm (log₁₀ CFU) in the presence exemplary PAA boosters containing glycolic acid per Example A, or containing lactic acid per Example O, are set forth.

TABLE 7
Reduction of P. aeurginosa biofilm (log₁₀ CFU) upon exposure to
PAA in the absence or presence of High Foam Booster¹ based on
Glycolic or Lactic Acids

| | Booster | | |
|---|---|---|---|
| | | Glycolic acid | Lactic acid |
| PAA (ppm) | None | (Example A) | (Example O) |
| 0 | NA² | 4.79 | 5.92 |
| 10 | 1.33 | 3.19 | 5.28 |
| 25 | 1.33 | 4.70 | 4.77 |
| 50 | 1.33 | 5.90 | >6.33 |
| 100 | 1.42 | >6.33 | >6.33 |
| 200 | 2.16 | >6.33 | >6.33 |

¹At 12.8 oz/gal Booster.
²Not applicable. Initial cell density was 7.33 log₁₀ CFU.

As can be seen in Table 7, when exemplary PA boosters are added to the PA use solutions, regardless of the primary acid that is present in the PA boosters, biofilm bacteria kill is approximately the same. Thus, it is believed that there is no notable difference in efficacy regardless of which primary acid is present in exemplary PA boosters.

Referencing Table 8, additional data is generated using various industry standards to demonstrate the boosting of the efficacy of Proxitane® EQ Peracetic Acid by an exemplary foaming PA booster pursuant to Example A and an exemplary no/low foaming PA booster pursuant to Example I (dose rates for foaming PA boosters are in rows 2-6 and dose rates for low/no foaming PA boosters are in rows 7-11).

TABLE 8

| ASTM/ | | | | | | | | | | | Primary | |
|---|---|---|---|---|---|---|---|---|---|---|---|---|
| AOAC | | | Dose Rate Oz | | | | | | | Pri- | + | Short |
| Method | | | (PAAPA | | | Result | Result | | Perox- | mary | Ancillary | Chain |
| Corrella- | | | Booster: | Contact | | w/PA | w/o PA | PAA | ide | Acid | acid | Acid |
| tion | Study | Organism | Water) | Time | Surface | Booster | Booster | ppm | ppm | ppm | ppm | ppm |
| AOAC | Disinfection | P. aeruginosa | 0.4:12.8:114.8 | 5 minute | Stainless | Pass | Mixed | 165 | 685 | 588 | 1088 | 1488 |
| 955.14; | | ATCC 15442, | (0.4 oz PAA, | | Steel | | | | | | | |
| 955.15; | | S. aureus | 12.8 oz PA | | | | | | | | | |
| 964.02 | | ATCC 6538, | Foaming | | | | | | | | | |
| | | S. enterica | Booster, | | | | | | | | | |
| | | ATCC 10708, | 114.8 oz | | | | | | | | | |
| | | S. enteritidis | Water) | | | | | | | | | |
| | | ATCC 13076, | | | | | | | | | | |
| | | E. coli O157: H7 | | | | | | | | | | |
| | | ATCC 35150, | | | | | | | | | | |
| | | L. monocytogenes | | | | | | | | | | |
| | | ATCC 19111, | | | | | | | | | | |
| | | C. jejuni | | | | | | | | | | |
| | | ATCC 29428, | | | | | | | | | | |
| | | C. Sakazakii | | | | | | | | | | |
| | | ATCC 29544, | | | | | | | | | | |
| | | E. coli O26; H11 | | | | | | | | | | |
| | | ATCC | | | | | | | | | | |
| ASTM | NFC | S. aureus | 0.2:12.8:115 | 5 minute | Glass | Pass | NA | 80 | 333 | 588 | 1088 | 1488 |
| E1153 | | ATCC 6538, | | | | | | | | | | |
| | | K. Aero 10348 | | | | | | | | | | |
| AOAC | Food | E. Coli 11229, | 0.2:12.8:115 | 0.5 minute | Glass | Pass | Pass | 80 | 333 | 588 | 1088 | 1488 |
| 960.09 | Contact | S. Aureus | | | | | | | | | | |
| | | ATCC 6538, | | | | | | | | | | |
| | | L. monocytogenes | | | | | | | | | | |
| | | ATCC 19111, | | | | | | | | | | |
| | | P. aeruginosa | | | | | | | | | | |
| | | ATCC 15442, | | | | | | | | | | |
| | | S. enterica | | | | | | | | | | |
| | | ATCC 10708 | | | | | | | | | | |
| ASTM | Biofilm Kill | P. aeruginosa | 2:12.8:113.8 | 10 minute | Borosilicate | Pass | Fail | 828 | 3400 | 588 | 1088 | 1488 |
| E2871-19 | | ATCC 15442, | | (5 min | Glass | | | | | | | |
| | | S. aureus | | staph) | | | | | | | | |
| | | ATCC 6538, | | | | | | | | | | |
| | | L. monocytogenes | | | | | | | | | | |
| | | ATCC 49594 | | | | | | | | | | |
| AOAC | Fungicidal | T. intergitales | 2:12.8:113.2 | 10 minute | Stainless | Pass | Fail | 828 | 3400 | 588 | 1088 | 1488 |
| 955.17 | | ATCC 9533 | | | Steel | | | | | | | |
| AOAC | Disinfection | P. aeruginosa | 0.5:0.5:127 | 5 minute | Stainless | Pass | Mixed | 205 | 750 | 391 | 490 | 646 |
| 955.14; | | ATCC 15442, | (0.5 oz PAA, | | Steel | | | | | | | |
| 955.15; | | S. aureus | 0.5 oz No/Low | | | | | | | | | |
| 964.02 | | ATCC 6538, | Foaming PA | | | | | | | | | |
| | | S. enterica | Booster: | | | | | | | | | |
| | | ATCC 10708, | 127 oz water) | | | | | | | | | |
| | | S. enteritidis | | | | | | | | | | |
| | | ATCC 13076, | | | | | | | | | | |
| | | E. coli O157: H7 | | | | | | | | | | |
| | | ATCC 35150, | | | | | | | | | | |
| | | L. monocytogenes | | | | | | | | | | |
| | | ATCC 19111, | | | | | | | | | | |
| | | C. jejuni | | | | | | | | | | |
| | | ATCC 29428, | | | | | | | | | | |
| | | C. Sakazakii | | | | | | | | | | |
| | | ATCC 29544 | | | | | | | | | | |
| AOAC | Food | E. Coli 11229, | 0.2:0.5:127.3 | 0.5 minute | Glass | Pass | Pass | 80 | 333 | 391 | 490 | 646 |
| 960.09 | Contact | S. Aureus | | | | | | | | | | |
| | | ATCC 6538, | | | | | | | | | | |
| | | L. monocytogenes | | | | | | | | | | |
| | | ATCC 19111, | | | | | | | | | | |
| | | P. aeruginosa | | | | | | | | | | |
| | | ATCC 15442, | | | | | | | | | | |
| | | S. enterica | | | | | | | | | | |
| | | ATCC 10708 | | | | | | | | | | |
| ASTM | Bioffilm KIll | P. aeruginosa | 2:0.5:125.5 | 10 minute | Borosilicate | Pass | Fail | 828 | 3400 | 391 | 490 | 646 |
| E2871-19 | | ATCC 15442, | | (5 min | Glass | | | | | | | |
| | | S. aureus | | staph) | | | | | | | | |
| | | ATCC 6538, | | | | | | | | | | |
| | | L. monocytogenes | | | | | | | | | | |
| | | ATCC 49594 | | | | | | | | | | |
| AOAC | Sporicidal | C. sporogenes | 2.5:0.5:125 | 10 minute | Porcelain | Pass | Fail | 828 | 3400 | 391 | 490 | 646 |
| 966.04 | | ATCC 3584, | | | Penicylinders | | | | | | | |
| | | B. Subtilis | | | | | | | | | | |
| | | ATCC 19659 | | | | | | | | | | |
| AOAC | Fungicidal | T. intergitales | 2:0.5:125.5 | 10 minute | Stainless | Pass | Fail | 828 | 3400 | 391 | 490 | 646 |
| 955.17 | | ATCC 9533 | | | Steel | | | | | | | |

As can be seen in Table 8, a use solution comprising Proxitane® EQ Peracetic Acid and water when applied to a variety of hard surfaces provides for mixed results at best, even when left on a hard surface for a contact time of up to 10 minutes. In comparison, when a use solution comprising an exemplary foaming or no/low foaming PA Booster, Proxitane® EQ Peracetic Acid and water is tested, all of the industry standard tests are passed, even at a contact time as short as 0.5 minutes, and even a biofilm kill in a time as short as 5 minutes or less.

The Minimum Biofilm Eradication Concentration (MBEC) Assay (ASTM E2799-17) [(approved Apr. 1, 2017 & published in May of 2017) is used to screen against biofilm formed by Pseudomonas aeruginosa ATCC 15442. Various concentrations of PAA+/— an exemplary PA booster (1:10) in 400 ppm hard water are tested with a 10-minute contact time. Further biofilm testing against P. aeruginosa is performed using the CDC Biofilm Reactor (ASTM E2871-12) [(approved Apr. 1, 2012 & published in June of 2012); with 304 stainless steel coupons and the Single Tube Method (ASTM E2871-21). Coupons are exposed to various concentrations of PAA+/— an exemplary PA booster (1:10) for 10 minutes. Imaging studies are performed. CDC coupons containing P. aeruginosa biofilm are exposed to hard water (control) or PAA plus the PA booster for 10 minutes with shear. After 1-3 exposures, the coupons are stained with LIVE/DEAD™ BacLight™ Bacterial Viability Kit stain and imaged using a Leica TCS-SP5 Confocal Scanning Laser Microscope.

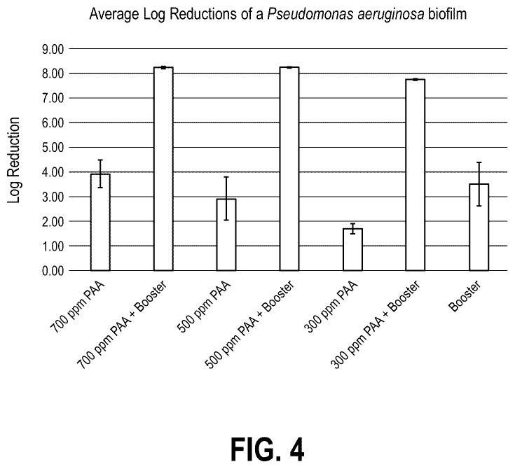

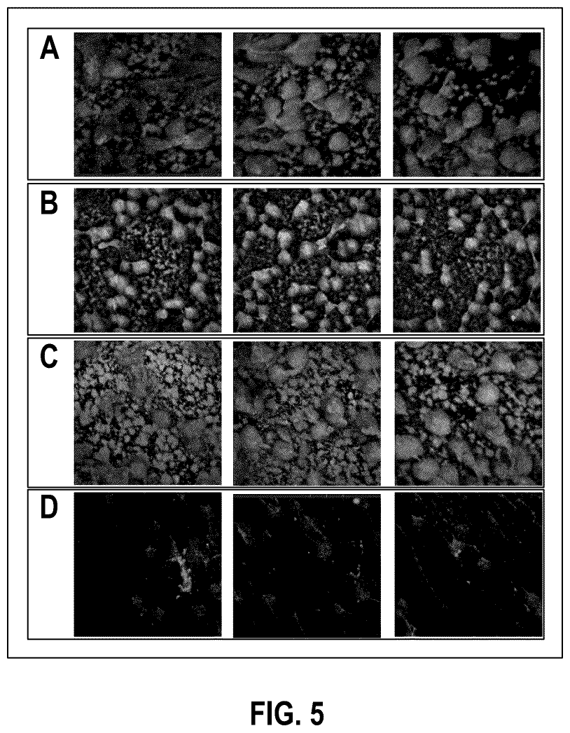

Results of the aforementioned testing are as follows. The combination of PAA and PA booster achieves consistent >6-log kill at 100 ppm PAA whereas PAA alone requires >400 ppm PAA. The PA booster alone achieves a 4-log kill. Referencing FIG. 4, the combination of PAA and an exemplary PA booster achieves >6-log₁₀ kill at 300 ppm PAA compared to PAA alone which requires >700 ppm. The PA booster alone achieves a 3 to 4-log₁₀ kill. Referencing FIG. 5, shown are confocal images of Pseudomonas aeruginosa biofilm after a 10-minute treatment at 20° C. with A) Hard water 1×, B) PAA with an exemplary PA Booster 1×, C) Hard water 3×, and D) PAA with PA Booster 3×. After one treatment of PAA in combination with the PA booster, the biofilm shows significant kill but appears to be intact. After three treatments of PAA in combination with the PA booster, the images indicate significant biofilm destruction.

The foregoing may be summarized as follows. An exemplary PA booster containing a mixture of organic acids, chelants, surfactants, and biodispersant is evaluated for its ability to enhance PAA performance against biofilm and endospores. In all testing performed, the exemplary PA booster is applied at a dilution of 1:10 in water regardless of the PAA concentration.

In both the MBEC and Single Tube Assays much lower levels of PAA combined with PA booster are needed to achieve 6-log reductions in biofilm cell counts compared to PAA alone. The PA booster alone achieves some level of log reduction (approx. 3-4) but is unable to generate 6-log reductions without PAA.

Confocal imaging is used to visualize the performance of PAA plus an exemplary PA booster against biofilm. After one treatment, significant kill is observed, but the biofilm appears to remain intact. After three treatments significant destruction of the biofilm becomes evident.

Endospores are highly resistant structures that form under stressful conditions.

Application of an exemplary PA booster alone shows little-to-no sporicidal activity against B. subtilis (data not shown) but does enhance the performance of PAA by 1- to 2-log reduction values depending on the PAA concentration. Against C. sporogenes spores, a >3-log reduction increase is observed at one concentration of PAA.

Bacterial endospores are dormant, highly resistant structures formed under stressful conditions. Endospores from Bacillus and Clostridium spp. are resistant to various environmental factors and some disinfectants. A sporicidal screening method to understand the efficacy of disinfectants against endospores was developed. The goal of the developed method was to show the benefit of using PAA in combination with a PAA booster.

Materials and Methods

Jars. 1 oz. (30 mL), recommended. Sterile.

Organisms. Spore preparations of Bacillus subtilis ATCC 6051 and Clostridium sporogenes ATCC 3584.

Circular stainless-steel carriers. 2 cm diameter discs, 304 grade with 2b finish on both sides, surface should be flat.

Hard water. 400 ppm hard water prepared as described in EPA SOP MB 30-2, which may be found at epa.gov/sites/default/files/2019-08/documents/mb-30-02.pdf.

Agar. Tryptic soy agar (TSA) and TSA+5% Sheep's Blood (bTSA).

Media. Reinforced Clostridial Media (RCM), Cooked Meat Media (CMM), Tryptic Soy Broth (TSB),

Diluent. Phosphate Buffer Dilution Water (PBDW).

Neutralizer. 2× Dey Engley+0.2% Catalase.

Soil. 5% Fetal Bovine Serum (FBS).

Organism Preparation

Bacillus subtilis. From a TSA stock plate inoculate a 10 ml TSB culture and incubate for 24 h at 35-37° C. Spread plate 0.5 ml of broth on each of 5 TSA plates. Incubate plates for 5 days at 35-37° C. Use 10 mL of PBDW for each plate and scrape growth off without disturbing agar and add to two 50 ml tubes (approx. 25 ml/tube). Centrifuge the tubes for 20 minutes at 5,000×g. Remove the supernatant and resuspend each pellet with 25 mL of PBDW. Split the 50 mL into five 50 ml tubes (approx. 10 ml each) and heat in a water bath at 80° C. for 15 minutes. Combine the tubes into two of the tubes (approx. 25 ml each) and centrifuge for 20 minutes at 5,000 ×g. Remove the supernatant and resuspend each pellet in 5 mL of PBDW, combine and store at 4-5° C. After 5 days at 4° C. plate the spore prep, remove 1 mL, and split into two aliquots. Heat one aliquot for 15 min at 80° C. Serially dilute both aliquots in PBDW and plate on TSA (5-6-7-8 plates). The first dilution 0.1 ml is added to 9.9 ml PBDW (10-2 dilution). After approx. 24 h at 35-37° C., record plate counts.

Clostridium sporogenes. Create (2) sterile tubes with 10 mL of RCM. Use 5-6 mL from one of the RCM tubes to rehydrate the lyophilized organism. Mix and aseptically transfer this aliquot back into the broth RCM tube. Once thoroughly vortexed, inoculate the second reinforced clostridial tube with 0.5 mL. Incubate tubes under anaerobic conditions at 36° C. for 24-48 hrs. Prepare CMM in tubes. After incubation, vortex and inoculate the CMM tubes with a loopful of C. sporogenes from the RCM tubes. Incubate the inoculated CMM under anaerobic conditions at 36° C. for 24-72 hours. After incubation, use cheese cloth or gauze to filter the CMM tubes collecting the supernatant in a 50 mL conical tube. Plate 0.1 mL aliquots of a 10⁻⁵, 10⁻⁶, and a 10⁻⁷ dilution on bTSA and incubate the plates under anaerobic conditions at 36° C. for 24-72 hours, record plate counts. Store the organism prep at 4-5° C.

Procedure

Prepare test inoculum as follows: 0.475 mL of organism prep combined with 0.025 mL of FBS. Place sterile carriers, using sterile forceps, into a 1 oz. jar and inoculate each with 0.02 mL of test inoculum. Spread the inoculum across the surface of the carrier using a pipette tip. Loosely place the lids onto the jars and place them into an incubator set to 36° C. with 40% RH. Allow carriers to dry for 60 minutes before testing. Following the dry time, remove the jars with the carriers and begin adding the test or control substance(s).

For controls, treat the carriers using hard water. For test carriers, use the test substance(s) for treatment. At time 0, using an interval of 15 or 30 seconds, add the control or test substance to the carrier by running the liquid down the side of the jar. Swirl the jar gently to evenly cover the carrier. After the contact time (i.e., 10 minutes), add 20 mL of neutralizer to the jars. Vortex the jars for 15 seconds before dilution plating. For control carriers, plate 0.1 mL aliquots of a 10-1, 10-2, and a 10-3 dilution on TSA for B. subtilis and bTSA for C. sporogenes. For test carriers plate the appropriate dilutions to observe organism growth.

Incubate plates for 24 hours aerobically for B. subtilis and 48 hours anaerobically for C. sporogenes at 36° C. Record counts and calculate log density for all controls and treatments.

Results:

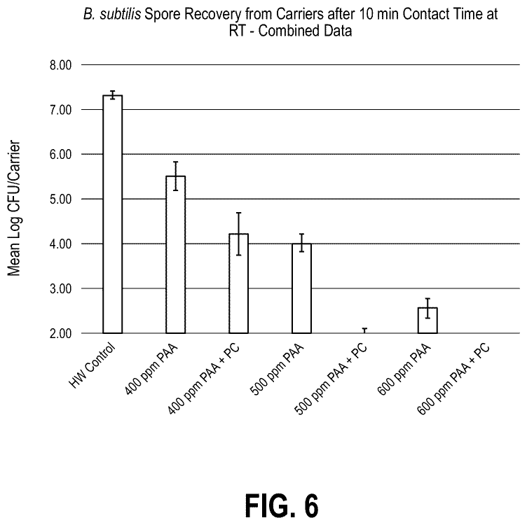

FIG. 6 shows that Bacillus subtilis ATCC 6051 recovered log densities after treatments with Hard Water, PAA alone at various concentrations, or PAA in combination with PA Booster at various concentrations at room temperature for 10 minutes.

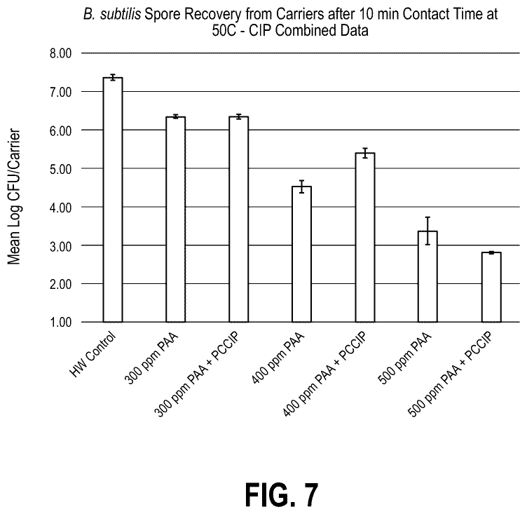

FIG. 7 shows that Bacillus subtilis ATCC 6051 recovered log densities after treatments with Hard water, PAA alone at various concentrations, or PAA in combination with PA Booster (CIP application) at various concentrations at 50° C. for 10 minutes.

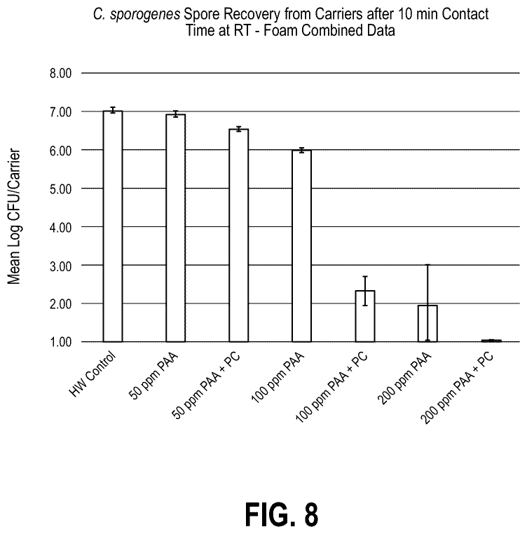

FIG. 8 shows that Clostridium sporogenes ATCC 3584 recovered log densities after treatments with Hard water, PAA alone at various concentrations, or PAA in combination with PA Booster at various concentrations at room temperature for 10 minutes.

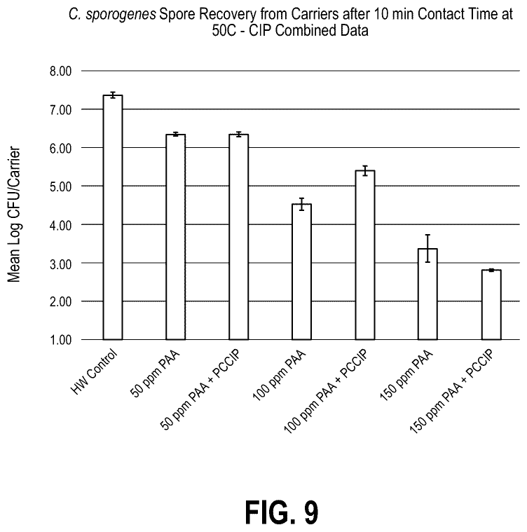

FIG. 9 shows that Clostridium sporogenes ATCC 3584 recovered log densities after treatments with Hard water, PAA alone at various concentrations, or PAA in combination with PA Booster (CIP application) at various concentrations at 50° C. for 10 minutes.

Discussion/Conclusions:

Against endospores dried on stainless steel carriers, PA Booster Foam increased the sporicidal activity of PAA as much as 1-3 log reduction values compared to PAA alone. Against C. sporogenes, ProvaCharge CIP at 100 ppm of PAA increased sporicidal activity of PAA by >3 log reduction. ProvaCharge CIP against B. subtilis did not show a significant benefit over PAA alone at all concentrations tested.

The results indicate that PA Booster can increase the performance of PAA against both B. subtilis and C. sporogenes endospores. Additionally, PA booster (via CIP application) can increase the performance of PAA against C. sporogenes endospores.

In light of the foregoing, it may be surmised that exemplary PA boosters can increase the performance of PAA against bacterial biofilm and endospores.

Additional Examples:

A first exemplary aqueous composition comprising:
a. a primary organic acid selected from: glycolic acid, benzoic acid, fumaric acid, caprylic acid, lactic acid, proprionic acid, a salt of any one of the foregoing organic acids and combinations thereof;
b. an ancillary organic acid that is different from the primary organic acid, the ancillary organic acid having a:
   i. carbon chain length of C6 or less, and
   ii. at least one pKa of from about 2 to about 7;
b. a biodispersant selected from: alkyl sulfosuccinate, alkyl sulfoacetate, alkylamide hydrolysate; a metal salt of any one the foregoing and combinations thereof
c. a chelant; and
e. a pH buffering agent;
wherein the aqueous composition has a pH of from about 3 to about 4.

A second exemplary composition according to the first exemplary composition, wherein the composition is a foaming composition that further comprises:
a. the primary organic acid at from about 0.1% to about 30% by weight of the aqueous composition; and
b. a foaming agent selected from: an ethoxylated alcohol, an amine oxide, a sulfonic acid and combinations thereof.

A third exemplary composition according to either one of the first and second exemplary compositions, wherein the primary organic acid is glycolic acid.

A fourth exemplary aqueous composition according to the third exemplary composition, wherein the glycolic acid is present in the aqueous composition at from about 0.1% to about 30% by weight of the aqueous composition.

A fifth exemplary composition according to any one of the preceding exemplary compositions, wherein the biodispersant is selected from: sodium dioctyl sulfosuccinate, disodium lauryl sulfosuccinate, sodium lauryl sulfoacetate and combinations thereof.

A sixth exemplary composition according to any one of the preceding exemplary compositions, wherein the biodispersant is sodium dioctyl sulfosuccinate.

A seventh exemplary composition according to the sixth exemplary composition, wherein the sodium dioctyl sulfosuccinate is present in the aqueous composition at from about 0.1% to about 10% by weight of the aqueous composition.

An eighth exemplary composition according to the first exemplary composition, wherein the composition is a low or no foaming composition that:
a. comprises the primary organic acid at from about 1% to about 15% by weight of the aqueous composition; and
b. further comprises:
   i. a defoamer; and
   ii. a coupling agent.

A ninth exemplary composition according to the eighth exemplary composition, wherein the primary organic acid is glycolic acid.

A tenth exemplary composition according to the ninth exemplary composition, wherein the glycolic acid is present in the aqueous composition at from about 1.0% to about 15% by weight of the aqueous composition.

An eleventh exemplary composition according to any one of the eighth through tenth exemplary compositions, wherein the biodispersant is selected from: sodium dioctyl sulfosuccinate, disodium lauryl sulfosuccinate, sodium lauryl sulfoacetate and combinations thereof.

A twelfth exemplary composition according to the eleventh exemplary composition, wherein the biodispersant is sodium dioctyl sulfosuccinate.

A thirteenth exemplary composition according to the twelfth exemplary composition, wherein the sodium dioctyl sulfosuccinate is present in the aqueous composition at from about 1.0% to about 10% by weight of the aqueous composition.

A fourteenth exemplary composition according to any one of the eighth through thirteenth exemplary compositions, wherein the defoamer is polyoxypropylene-polyethylene block copolymer and is present in the aqueous composition at from about 4% to about 15% by weight of the aqueous composition.

A fifteenth exemplary composition according to any one of the eighth through fourteenth exemplary compositions, wherein the coupling agent is iminodipropate alanate that is present in the aqueous composition at from about 1% to about 10% by weight of the aqueous composition.

A sixteenth exemplary clear aqueous composition consisting of:
a. at least one organic acid selected from:
   i. glycolic acid,
   ii. glycolic acid and an acid selected from: benzoic acid, a metal salt of benzoic acid, fumaric acid, a metal salt of fumaric acid, caprylic acid, a metal salt of caprylic acid, lactic acid, a metal salt of lactic acid, proprionic acid, a metal salt of proprionic acid and combinations thereof, or
   iii. at least two organic acids selected from: benzoic acid, a metal salt of benzoic acid, fumaric acid, a metal salt of fumaric acid, caprylic acid, a metal salt of caprylic acid, lactic acid, a metal salt of lactic acid, proprionic acid, a metal salt of proprionic;
b. a biodispersant selected from: a sodium salt of an alkyl sulfosuccinate, a sodium salt of an alkyl sulfoacetate and combinations thereof;
c. a chelant;
d. an acidifying agent;
e. a pH buffering agent; and
f. optionally:
   i. a foaming agent selected from: an ethoxylated alcohol, an amine oxide, a dodecylbenzene sulfonic acid and combinations thereof; or
   ii. a defoamer and a coupling agent;
wherein the aqueous composition has a pH of from about 3 to about 4.

A seventeenth exemplary composition according to any one of the first through sixteenth exemplary compositions, wherein the exemplary composition is substantially free of one, two or all three of the following:
i. peracids,
ii. peroxides,
wherein the peroxides may be selected from: organic peroxides, inorganic peroxides and combinations thereof, and
iii. inorganic acids.

A first exemplary method of boosting biofilm kill of a peracid disinfectant/sanitizer use solution made from a peracid containing disinfectant/sanitizer concentrate, wherein:
(a) a first peracid disinfectant/sanitizer use solution:
   i. comprises the aqueous peracid disinfectant/sanitizer concentrate and water,
   ii. contains less than 800 ppm total of peracids selected from: peroxyacetic acid; peroxyoctanoic acid; peroxysulfonic acid; peroxyformic acid; and combinations thereof,
   iii. has a pH of from about 1 to about 5, and
   iv. is characterized by a first efficacy of equal to or less than about a 5 log₁₀ reduction of a Pseudomonas aeruginosa biofilm after contact therewith for 10 minutes or more pursuant to ASTM E2871-12 (approved Apr. 1, 2012 & published in June of 2012); and
(b) a second peracid disinfectant/sanitizer use solution:
   i. comprises a composition according to any one of the first through seventeenth exemplary compositions, the aqueous peracid disinfectant/sanitizer concentrate and water,
   ii. contains less than 800 ppm total of peracids selected from: peroxyacetic acid; peroxyoctanoic acid; peroxysulfonic acid; peroxyformic acid; and combinations thereof;
   iii. has a pH of from about 1 to about 5, and
   iv. is characterized by a boosted efficacy equal to or greater than 6 log₁₀ reduction of Pseudomonas aeruginosa biofilm after contact therewith for 10 minutes or more pursuant to ASTM E2871-12 (approved Apr. 1, 2012 & published in June of 2012);
the method comprising:
   (a) making the second peracid disinfectant/sanitizer use solution by mixing together any one of the first through seventeenth exemplary compositions, the aqueous peracid disinfectant/sanitizer concentrate and water, and
   (b) applying the second peracid disinfectant/sanitizer use solution to a hard surface.

A second exemplary method of boosting an efficacy of a concentrated disinfectant/sanitizer that comprises one or more peracids at a total weight percentage of 800 ppm or less by weight of the concentrated disinfectant/sanitizer, the method comprising making a use solution by mixing together the concentrated disinfectant/sanitizer, water and an exemplary composition according to any one of the first through seventeenth exemplary compositions;
wherein:
   (a) a use solution consisting of the concentrated disinfectant/sanitizer diluted with water has a first efficacy,
   (b) the use solution of (a) has a second efficacy, and
   (c) the second efficacy is from about 2 log₁₀ to about 5 log₁₀ cycles greater than the first efficacy against Pseudomonas aeruginosa after contact therewith for 10 minutes or more pursuant to ASTM E2871-19 (approved Jan. 1, 2019 & published in February of 2019).

A first exemplary kit comprising:
(a) a first container containing any one of the first through seventeenth exemplary compositions; and
(b) a second container containing an aqueous composition comprising peracetic acid.

A second exemplary kit according to the first exemplary kit, wherein the aqueous composition comprising peracetic acid:
(a) contains from about 4% to about 6% by weight of peracetic acid; and
(b) is substantially free of surfactant.

Having shown and described various embodiments of the present invention, further adaptations of the methods and systems described herein may be accomplished by appropriate modifications by one of ordinary skill in the art without departing from the scope of the present invention. Several such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of any claims that may be presented and is understood not to be limited to the details of structure and operation shown and described in the specification and drawings.

What is claimed is:

1. A method for disinfecting, sterilizing, and/or sanitizing a surface intended for human or veterinary medical use, comprising contacting said surface with mixture comprising
a. a peracid-containing composition and
b. a peracid (PA) boosting composition, said PA boosting composition comprising a primary acid, a biodispersant, a chelant, a pH buffering agent, a foaming agent, a defoaming agent, and a coupling agent.

2. The method of claim 1 wherein said surface is a surface of a hospital room, said surface being selected from a floor, a wall, a ceiling, a window, a table, a sink, a bedrail, a counter, medical equipment, and combinations thereof.

3. The method of claim 1, wherein said disinfecting, sterilizing, and/or sanitizing achieves one or more of reducing, deactivating, removing, and/preventing a virus, an endospore, a bacterial, and combinations thereof.

4. The method of claim 1, wherein said disinfecting, sterilizing, and/or sanitizing reduces, deactivates, removes, and/or prevents a biofilm.

5. The method of claim 4, wherein said biofilm comprises an organism according to Table 8.

6. The method of claim 1 wherein said mixture comprises from about 10 ppm to 1000 ppm peracid.

7. The method claim 1, wherein said peracid is peracetic acid.

8. The method of claim 1, wherein said mixture comprises at least 300 ppm of said primary acid.

9. The method of claim 1, wherein said mixture further comprises an ancillary acid, and wherein said combined primary acid and ancillary acid is at least 500 ppm of said mixture.

10. The method of claim 1, wherein said PA booster further comprises an ancillary acid, and wherein primary acid is different from said ancillary acid.

11. The method of claim 1, wherein said primary acid is selected from glycolic acid, benzoic acid, fumaric acid, caprylic acid, lactic acid, propionic acid, and combinations thereof.

12. The method of claim 1, wherein said PA booster comprises from about 0.1% to about 30%, or from about 0.5% to about 20%, or from about 1.0% to about 15% of one or more primary organic acid.

13. The method of claim 1, wherein said PA boosting composition is characterized as low/no foaming.

14. The method of claim 1, wherein said PA boosting composition is characterized as foaming.

15. The method of claim 10, wherein said ancillary acid has a carbon chain length of C6 or less and at least one pKa of from about 2 to about 7.

16. The method of claim 10, wherein said ancillary acid is selected from acetic acid, formic acid, maleic acid, tartaric acid, and combinations thereof.

17. The method of claim 10, wherein said PA boosting composition comprises from about 0.1% to about 20% of said ancillary organic acid.

18. The method of claim 1, wherein said biodispersant is selected from alkyl sulfosuccinate, alkyl sulfoacetate, alkylamide hydrolysate, and combinations thereof.

19. The method of claim 1, wherein said PA boosting composition comprises from about 0.1% to about 10% of said biodispersant.

20. The method of claim 1, wherein said chelant is selected from citric acid, 1-hydroxyethylidene 1,1-diphosphonic acid (“HEDP”), trisodium salt of methylglycinediacetic acid, L-glutamic acid N,N-diacetic acid, tetrasodium salt, hydroxyethylene-diaminetriacetic acid, trisodium salt, a salt of ethylenediamine tetracetic acid (“EDTA”), and combinations thereof.

21. The method of claim 1, wherein said PA boosting composition has a pH of from about 3 to about 4, or from about 3.5 to about 4.

22. The method of claim 1, wherein said PA boosting composition comprises an ethoxylated alcohol selected from one or both of an amine oxide, and a sulfonic acid.

23. A method for disinfecting and/or sanitizing a surface intended for manufacture of a cosmetic, comprising contacting said surface with a peracid-containing composition and a peracetic acid (PAA) boosting composition, said PAA boosting composition comprising an organic acid, a biodispersant, a chelant, a pH buffering agent, a foaming agent, a defoaming agent, a coupling agent, and combinations thereof.

24. A method for disinfecting and/or sanitizing on a surface intended for manufacture of a pharmaceutical, comprising contacting said surface with a peracid-containing composition and a peracetic acid (PAA) boosting composition, said PAA boosting composition comprising an organic acid, a biodispersant, a chelant, a pH buffering agent, a foaming agent, a defoaming agent, a coupling agent, and combinations thereof.
